(19) United States
(12) Patent Application Publication
(10) Pub. No.: US 2021/0293744 A1
(43) Pub. Date: Sep. 23, 2021

(54) GAS SENSOR

(71) Applicant: NGK INSULATORS, LTD., Nagoya (JP)

(72) Inventors: Daichi ICHIKAWA, Nagoya (JP); Yuichiro KONDO, Obu (JP); Nobuhiko MORI, Nagoya (JP)

(21) Appl. No.: 17/205,602

(22) Filed: Mar. 18, 2021

(30) Foreign Application Priority Data
Mar. 19, 2020 (JP) 2020-049882
Oct. 13, 2020 (JP) 2020-172383

(51) Int. Cl. G01N 27/409; G01N 33/00; G01N 27/407; F01N 3/20

(52) U.S. Cl. CPC G01N 27/409; F01N 3/208; G01N 27/4073; G01N 33/0037

(57) ABSTRACT

A gas sensor that measures concentrations of a first target component and a second target component includes a first switch that controls driving of a first preliminary adjustment pump cell to be turned ON or OFF, a second switch that controls driving of a second preliminary adjustment pump cell to be turned ON or OFF, and a switching control device that controls switching between the first switch and the second switch.

### CROSS-REFERENCE TO RELATED APPLICATIONS

This application is based upon and claims the benefit of priority from Japanese Patent Applications No. 2020-049882 filed on Mar. 19, 2020 and No. 2020-172383 filed on Oct. 13, 2020, the contents all of which are incorporated herein by reference.

### BACKGROUND OF THE INVENTION

### Field of the Invention

The present invention relates to a gas sensor, which is capable of measuring respective concentrations of a plurality of target components within a gas to be measured.

### Description of the Related Art

Conventionally, gas sensors are know that serve to detect NOx and NH₃ (for example, refer to Japanese Laid-Open Patent Publication No. 2001-133447). More specifically, in Japanese Laid-Open Patent Publication No. 2001-133447, it is disclosed that a first gas sensor having a pump that is ON at all times, and a second gas sensor having a pump that is OFF at all times are used in order to detect NOx and NH₃.

### SUMMARY OF THE INVENTION

However, as disclosed in Japanese Laid-Open Patent Publication No. 2001-133447, in the case of using a first gas sensor having a pump that is ON at all times and a second gas sensor having a pump that is OFF at all times, electrode deterioration of the first gas sensor having the pump that is ON at all times disadvantageously progresses more so than that of the second gas sensor, so that when considered in a comprehensive manner, there is a concern that the useful lifetime of the gas sensor may be shortened.

The present invention has been devised taking into consideration the aforementioned problems, and has the object of providing a gas sensor, in which it is possible to accurately measure over a prolonged time period the concentration of a non-combustible component such as exhaust gas, and a plurality of components (for example, NO, NO₂, and NH₃) that coexist in the presence of oxygen.

A gas sensor according to one aspect of the present invention is a gas sensor configured to measure concentrations of a first target component and a second target component, including:

at least one sensor element;

a temperature control device configured to control a temperature of the sensor element;

at least one oxygen concentration control device; and

a target component concentration acquisition device;

wherein the sensor element includes a structural body made up from at least an oxygen ion conductive solid electrolyte, and at least one sensor cell formed in the structural body;

the sensor cell is equipped, in a direction in which a gas is introduced, with a gas introduction port, a first diffusion rate control member, a first chamber, a second diffusion rate control member, a second chamber, a third diffusion rate control member, and a measurement chamber;

the measurement chamber of the at least one sensor cell is equipped with target component measurement pump cells;

the oxygen concentration control device controls oxygen concentrations of the first chamber and the second chamber of the at least one sensor cell; and

in the target component concentration acquisition device:

a concentration of the second target component is acquired on a basis of a difference between a current value flowing to one of the target component measurement pump cells, and a current value flowing to another one of the target component measurement pump cells;

a total concentration of the first target component and the second target component is acquired from the current value flowing to the another one of the target component measurement pump cells; and

a concentration of the first target component is acquired by subtracting the concentration of the second target component from the total concentration.

In accordance with the gas sensor according to the present invention, it is possible to accurately measure over a prolonged time period the concentration of a non-combustible component such as exhaust gas, and a plurality of components (for example, NO, NO₂, and NH₃) that coexist in the presence of oxygen.

The above and other objects, features, and advantages of the present invention will become more apparent from the following description when taken in conjunction with the accompanying drawings, in which preferred embodiments of the present invention are shown by way of illustrative example.

### BRIEF DESCRIPTION OF THE DRAWINGS

FIG. 1 is a cross-sectional view in which there is shown one structural example of a gas sensor according to an embodiment of the present invention (a cross-sectional view taken along line I-I in FIGS. 2 and 3: dashed lines omitted);

FIG. 2 is a cross-sectional view (a cross-sectional view taken along line II-II in FIG. 1) showing a structural example of a first sensor cell of the gas sensor;

FIG. 3 is a cross-sectional view (a cross-sectional view taken along line III-III in FIG. 1) showing a structural example of a second sensor cell of the gas sensor;

FIG. 4 is a configuration diagram schematically showing the gas sensor;

FIG. 5 is an explanatory diagram schematically showing reactions inside a first preliminary adjustment chamber, inside a first oxygen concentration adjustment chamber, and inside a first measurement chamber of a first sensor cell, as well as reactions inside a second preliminary adjustment chamber, a second oxygen concentration adjustment chamber, and inside a second measurement chamber of a second sensor cell, in the case that a first preliminary adjustment pump cell is turned ON, and a second preliminary adjustment pump cell is turned OFF;

FIG. 6 is an explanatory diagram schematically showing reactions inside a first preliminary adjustment chamber, inside a first oxygen concentration adjustment chamber, and inside a first measurement chamber of a first sensor cell, as well as reactions inside a second preliminary adjustment chamber, a second oxygen concentration adjustment chamber, and inside a second measurement chamber of a second sensor cell, in the case that the first preliminary adjustment pump cell is turned OFF, and the second preliminary adjustment pump cell is turned ON;

FIG. 7 is a graph showing a map utilized by the gas sensor;

FIG. 8 is an explanatory diagram showing the map utilized by the gas sensor in the form of a table;

FIG. 9 is an explanatory diagram showing measurement results in the form of a table in order to confirm the certainty of the map;

FIG. 10A is a timing chart showing the start of operation and an end of operation of a vehicle or the like at a first switching timing;

FIG. 10B is a timing chart showing an ON/OFF switching timing of a first preliminary adjustment pump cell and a second preliminary adjustment pump cell;

FIG. 10C is a block diagram of a switching control;

FIG. 11 is a flowchart showing an ON/OFF switching timing of a first preliminary adjustment pump cell and a second preliminary adjustment pump cell at the first switching timing;

FIG. 12A is a timing chart showing a start of operation and an end of operation of a vehicle or the like at a second switching timing;

FIG. 12B is a timing chart showing an ON/OFF switching timing of a first preliminary adjustment pump cell and a second preliminary adjustment pump cell;

FIG. 12C is a block diagram of a switching control;

FIG. 13 is a flowchart showing an ON/OFF switching timing of a first preliminary adjustment pump cell and a second preliminary adjustment pump cell at the second switching timing;

FIG. 14A is a timing chart showing a start of operation and an end of operation of a vehicle or the like at a third switching timing;

FIG. 14B is a timing chart showing an ON/OFF switching timing of a first preliminary adjustment pump cell and a second preliminary adjustment pump cell;

FIG. 14C is a block diagram of a switching control;

FIG. 15 is a flowchart showing an ON/OFF switching timing of a first preliminary adjustment pump cell and a second preliminary adjustment pump cell at the third switching timing;

FIG. 16A is a timing chart showing a start of operation and an end of operation of a vehicle or the like at a fourth switching timing;

FIG. 16B is a timing chart showing an ON/OFF switching timing of a first preliminary adjustment pump cell and a second preliminary adjustment pump cell;

FIG. 16C is a block diagram of a switching control;

FIG. 17 is a flowchart showing an ON/OFF switching timing of a first preliminary adjustment pump cell and a second preliminary adjustment pump cell at the fourth switching timing;

FIG. 18 is a cross-sectional view showing a structural example of a first modification of the gas sensor; and

FIG. 19 is a cross-sectional view showing a structural example of a second modification of the gas sensor.

### DESCRIPTION OF THE PREFERRED EMBODIMENTS

A preferred embodiment of a metal terminal according to the present invention will be presented and described in detail below with reference to the accompanying drawings.

Initially, a basic exemplary configuration and measurement principles of a gas sensor 10 according to the present embodiment will be described below.

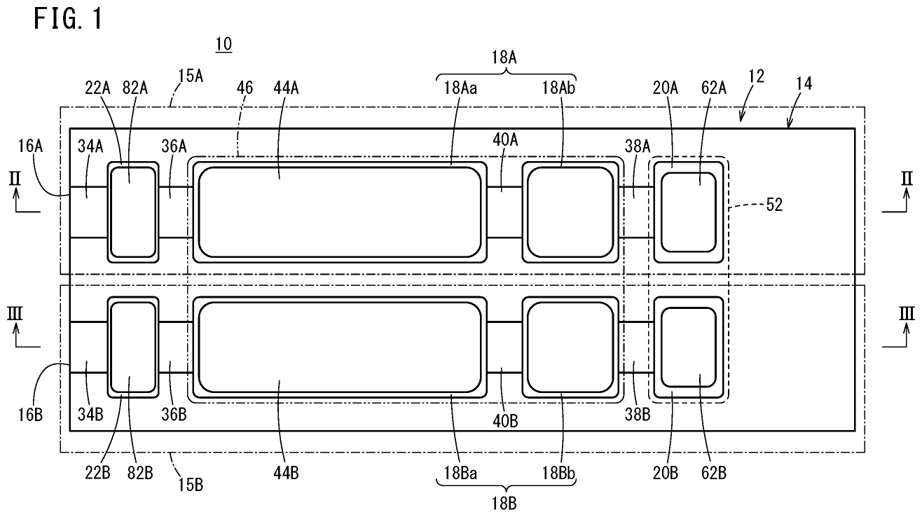

As shown in FIGS. 1 to 3, the gas sensor 10 includes a sensor element 12. The sensor element 12 includes a structural body 14 made up from an oxygen ion conductive solid electrolyte, and a first sensor cell 15A and a second sensor cell 15B formed in the structural body 14. Of course, a structure may be provided in which, from among two structural bodies 14, the first sensor cell 15A is formed in one of the structural bodies 14, and the second sensor cell 15B is formed in another one of the structural bodies 14. Such a configuration will be described later.

In this instance, when a thickness direction of the structural body 14 is defined as a vertical direction and a widthwise direction of the structural body 14 is defined as a horizontal direction, within the structural body 14, the first sensor cell 15A and the second sensor cell 15B are disposed in a state of being aligned in the horizontal direction.

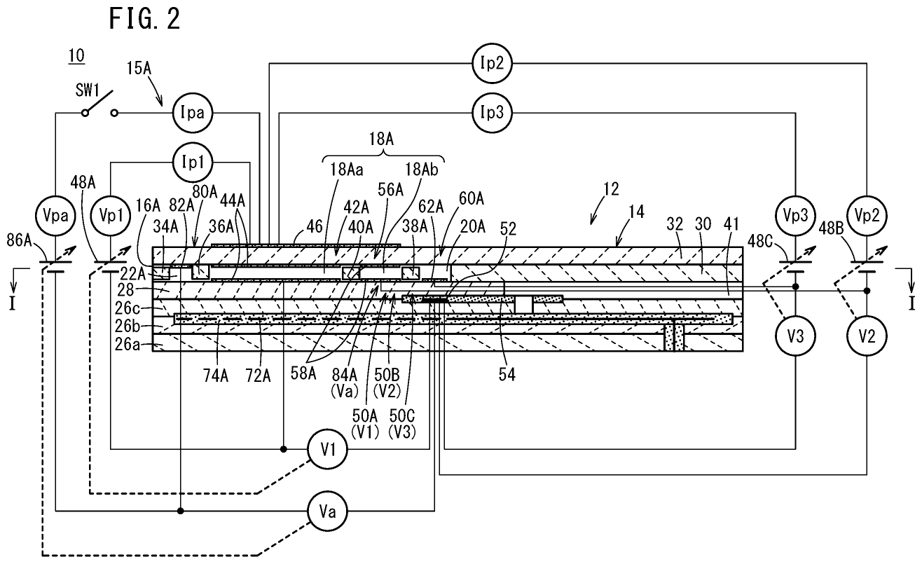

As shown in FIGS. 1 and 2, the first sensor cell 15A includes a first gas introduction port 16A formed in the structural body 14 and into which a gas to be measured is introduced, a first oxygen concentration adjustment chamber 18A formed inside the structural body 14 and communicating with the first gas introduction port 16A, and a first measurement chamber 20A formed inside the structural body 14 and communicating with the first oxygen concentration adjustment chamber 18A.

The first oxygen concentration adjustment chamber 18A includes a first main adjustment chamber 18Aa in communication with the first gas introduction port 16A, and a first auxiliary adjustment chamber 18Ab in communication with the first main adjustment chamber 18Aa. The first measurement chamber 20A communicates with the first auxiliary adjustment chamber 18Ab.

Furthermore, the first sensor cell 15A includes a first preliminary adjustment chamber 22A provided between the first gas introduction port 16A and the first main adjustment chamber 18Aa within the structural body 14, and which communicates with the first gas introduction port 16A.

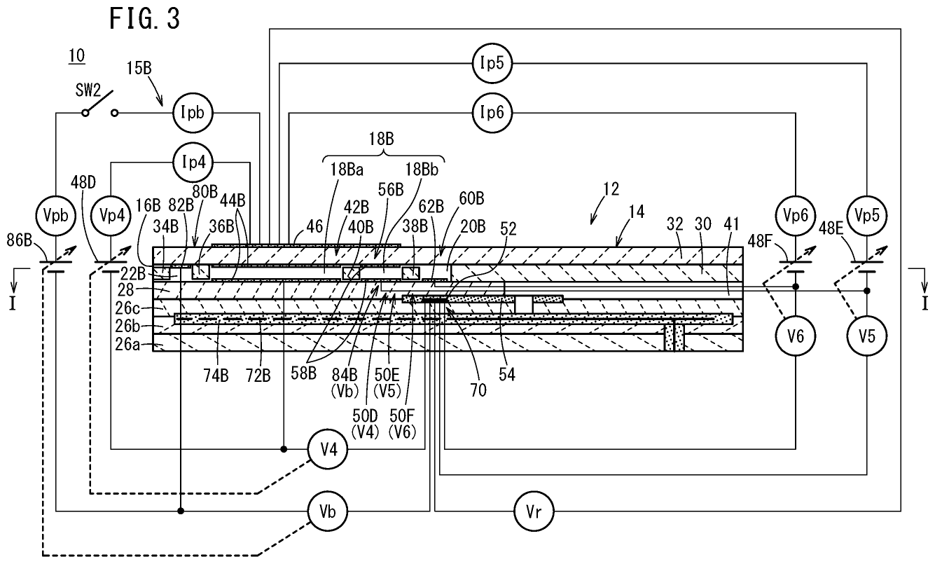

On the other hand, as shown in FIGS. 1 and 3, the second sensor cell 15B includes a second gas introduction port 16B formed in the structural body 14 and into which a gas to be measured is introduced, a second oxygen concentration adjustment chamber 18B formed inside the structural body 14 and communicating with the second gas introduction port 16B, and a second measurement chamber 20B formed inside the structural body 14 and communicating with the second oxygen concentration adjustment chamber 18B.

The second oxygen concentration adjustment chamber 18B includes a second main adjustment chamber 18Ba in communication with the second gas introduction port 16B, and a second auxiliary adjustment chamber 18Bb in communication with the second main adjustment chamber 18Ba. The second measurement chamber 20B communicates with the second auxiliary adjustment chamber 18Bb.

Furthermore, the second sensor cell 15B includes a second preliminary adjustment chamber 22B provided between the second gas introduction port 16B and the second main adjustment chamber 18Ba within the structural body 14, and which communicates with the second gas introduction port 16B.

As shown in FIGS. 2 and 3, the structural body 14 is constituted by six layers including a first substrate layer 26a, a second substrate layer 26b, a third substrate layer 26c, a first solid electrolyte layer 28, a spacer layer 30, and a second solid electrolyte layer 32, which are stacked in this order from a lower side as viewed in the drawing. The respective layers are composed respectively of an oxygen ion conductive solid electrolyte layer such as zirconia (ZrO₂) or the like.

As shown in FIG. 2, in the first sensor cell 15A, between a lower surface of the second solid electrolyte layer 32 and an upper surface of the first solid electrolyte layer 28 on a distal end side of the sensor element 12, there are provided the first gas introduction port 16A, a first diffusion rate control member 34A, the first preliminary adjustment chamber 22A, a second diffusion rate control member 36A, the first oxygen concentration adjustment chamber 18A, a third diffusion rate control member 38A, and the first measurement chamber 20A. Further, a fourth diffusion rate control member 40A is provided between the first main adjustment chamber 18Aa and the first auxiliary adjustment chamber 18Ab that make up the first oxygen concentration adjustment chamber 18A.

The first gas introduction port 16A, the first diffusion rate control member 34A, the first preliminary adjustment chamber 22A, the second diffusion rate control member 36A, the first main adjustment chamber 18Aa, the fourth diffusion rate control member 40A, the first auxiliary adjustment chamber 18Ab, the third diffusion rate control member 38A, and the first measurement chamber 20A are formed adjacent to each other in a manner communicating in this order. A portion from the first gas introduction port 16A leading to the first measurement chamber 20A may also be referred to as a first gas flow section.

The first gas introduction port 16A, the first preliminary adjustment chamber 22A, the first main adjustment chamber 18Aa, the first auxiliary adjustment chamber 18Ab, and the first measurement chamber 20A are internal spaces provided by hollowing out the spacer layer 30. Any of the first preliminary adjustment chamber 22A, the first main adjustment chamber 18Aa, the first auxiliary adjustment chamber 18Ab, and the first measurement chamber 20A is arranged in a manner so that respective upper parts thereof are defined by a lower surface of the second solid electrolyte layer 32, respective lower parts thereof are defined by an upper surface of the first solid electrolyte layer 28, and respective side parts thereof are defined by side surfaces of the spacer layer 30.

Similarly, in relation to the second sensor cell 15B as well, as shown in FIG. 3, between a lower surface of the second solid electrolyte layer 32 and an upper surface of the first solid electrolyte layer 28 on a distal end side of the sensor element 12, there are provided the second gas introduction port 16B, a first diffusion rate control member 34B, the second preliminary adjustment chamber 22B, a second diffusion rate control member 36B, the second oxygen concentration adjustment chamber 18B, a third diffusion rate control member 38B, and the second measurement chamber 20B. Further, a fourth diffusion rate control member 40B is provided between the second main adjustment chamber 18Ba and the second auxiliary adjustment chamber 18Bb that make up the second oxygen concentration adjustment chamber 18B.

The second gas introduction port 16B, the first diffusion rate control member 34B, the second preliminary adjustment chamber 22B, the second diffusion rate control member 36B, the second main adjustment chamber 18Ba, the fourth diffusion rate control member 40B, the second auxiliary adjustment chamber 18Bb, the third diffusion rate control member 38B, and the second measurement chamber 20B are formed adjacent to each other in a manner communicating in this order. A portion from the second gas introduction port 16B leading to the second measurement chamber 20B may also be referred to as a second gas flow section.

The second gas introduction port 16B, the second preliminary adjustment chamber 22B, the second main adjustment chamber 18Ba, the second auxiliary adjustment chamber 18Bb, and the second measurement chamber 20B are internal spaces provided by hollowing out the spacer layer 30. Any of the second preliminary adjustment chamber 22B, the second main adjustment chamber 18Ba, the second auxiliary adjustment chamber 18Bb, and the second measurement chamber 20B is arranged in a manner so that respective upper parts thereof are defined by a lower surface of the second solid electrolyte layer 32, respective lower parts thereof are defined by an upper surface of the first solid electrolyte layer 28, and respective side parts thereof are defined by side surfaces of the spacer layer 30.

Together with the first sensor cell 15A and the second sensor cell 15B, any of the first diffusion rate control members (34A and 34B), the third diffusion rate control members (38A and 38B), and the fourth diffusion rate control members (40A and 40B) are provided as one or two horizontally elongated slits (in which openings thereof have a longitudinal direction in a direction perpendicular to the drawing). The respective second diffusion rate control members (36A and 36B) are provided as one or two horizontally elongated slits (in which an opening thereof has a longitudinal direction in a direction perpendicular to the drawing).

Further, a reference gas introduction space 41, which is common to the first sensor cell 15A and the second sensor cell 15B, is disposed between the upper surface of the third substrate layer 26c and the lower surface of the spacer layer 30, at a position that is farther from the distal end side than the first gas flow section and the second gas flow section, respectively. The reference gas introduction space 41 is an internal space in which an upper part thereof is defined by a lower surface of the spacer layer 30, a lower part thereof is defined by an upper surface of the third substrate layer 26c, and side parts thereof are defined by side surfaces of the first solid electrolyte layer 28. For example, oxygen or atmospheric air is introduced as a reference gas into the reference gas introduction space 41.

The first gas introduction port 16A and the second gas introduction port 16B are locations that open with respect to the external space, and the gas to be measured is drawn into the first sensor cell 15A and the second sensor cell 15B from the external space through the first gas introduction port 16A and the second gas introduction port 16B.

The first diffusion rate control member 34A of the first sensor cell 15A is a location that imparts a predetermined diffusion resistance to the gas to be measured which is introduced from the first gas introduction port 16A into the first preliminary adjustment chamber 22A. The first diffusion rate control member 34B of the second sensor cell 15B is a location that imparts a predetermined diffusion resistance to the gas to be measured which is introduced from the second gas introduction port 16B into the second preliminary adjustment chamber 22B. The first preliminary adjustment chamber 22A and the second preliminary adjustment chamber 22B will be described later.

The second diffusion rate control member 36A of the first sensor cell 15A is a location that imparts a predetermined diffusion resistance to the gas to be measured which is introduced from the first preliminary adjustment chamber 22A into the first main adjustment chamber 18Aa. The second diffusion rate control member 36B of the second sensor cell 15B is a location that imparts a predetermined diffusion resistance to the gas to be measured which is introduced from the second preliminary adjustment chamber 22B into the second main adjustment chamber 18Ba.

The first main adjustment chamber 18Aa is provided as a space for the purpose of adjusting an oxygen partial pressure within the gas to be measured that is introduced from the first gas introduction port 16A. The oxygen partial pressure is adjusted by operation of a first main pump cell 42A. The second main adjustment chamber 18Ba is provided as a space for the purpose of adjusting an oxygen partial pressure within the gas to be measured that is introduced from the second gas introduction port 16B. The oxygen partial pressure is adjusted by operation of a second main pump cell 42B.

The first main pump cell 42A comprises a first electrochemical pump cell (main electrochemical pumping cell), which is constituted by including a first main interior side pump electrode 44A, an exterior side pump electrode 46 which is common to the first sensor cell 15A and the second sensor cell 15B, and an oxygen ion conductive solid electrolyte which is sandwiched between the two pump electrodes. The first main interior side pump electrode 44A is provided substantially over the entire surface, respectively, of an upper surface of the first solid electrolyte layer 28, a lower surface of the second solid electrolyte layer 32, and side surfaces of the spacer layer 30 that define the first main adjustment chamber 18Aa. The common exterior side pump electrode 46 extends, on the upper surface of the second solid electrolyte layer 32, from a region corresponding to the first main interior side pump electrode 44A to a region corresponding to a second main interior side pump electrode 44B (the second sensor cell 15B), and is provided in a form of being exposed to the external space.

The first main pump cell 42A applies a first pump voltage Vp1 supplied from a first variable power source 48A for the first sensor cell which is provided externally of the sensor element 12, and by allowing a first pump current Ip1 to flow between the common exterior side pump electrode 46 and the first main interior side pump electrode 44A, it is possible to pump oxygen in the interior of the first main adjustment chamber 18Aa into the external space, or alternatively, to pump oxygen in the external space into the first main adjustment chamber 18Aa.

Further, the first sensor cell 15A includes a first oxygen partial pressure detecting sensor cell 50A which is an electrochemical sensor cell. The first oxygen partial pressure detecting sensor cell 50A is constituted by the first main interior side pump electrode 44A, a common reference electrode 52 sandwiched between the first solid electrolyte layer 28 and an upper surface of the third substrate layer 26c, and an oxygen ion conductive solid electrolyte sandwiched between these electrodes. The common reference electrode 52 is an electrode having a substantially rectangular shape as viewed in plan, which is made from a porous cermet in the same manner as the common exterior side pump electrode 46 and the like. Further, around the periphery of the common reference electrode 52, a common reference gas introduction layer 54 is provided, which is made from porous alumina, and moreover, is connected to the common reference gas introduction space 41. More specifically, the reference gas in the reference gas introduction space 41 is introduced to the surface of the reference electrode 52 via the reference gas introduction layer 54. The first oxygen partial pressure detecting sensor cell 50A generates a first electromotive force V1 between the first main interior side pump electrode 44A and the reference electrode 52, which is caused by a difference in oxygen concentration between the atmosphere inside the first main adjustment chamber 18Aa and the reference gas in the reference gas introduction space 41.

The first electromotive force V1 generated in the first oxygen partial pressure detecting sensor cell 50A changes depending on the oxygen partial pressure of the atmosphere existing in the first main adjustment chamber 18Aa. In accordance with the aforementioned first electromotive force V1, the first sensor cell 15A feedback-controls the first variable power source 48A of the first main pump cell 42A. Consequently, the first pump voltage Vp1, which is applied by the first variable power source 48A to the first main pump cell 42A, can be controlled in accordance with the oxygen partial pressure of the atmosphere in the first main adjustment chamber 18Aa.

The fourth diffusion rate control member 40A imparts a predetermined diffusion resistance to the gas to be measured, the oxygen concentration (oxygen partial pressure) of which is controlled by operation of the first main pump cell 42A in the first main adjustment chamber 18Aa, and is a location that guides the gas to be measured into the first auxiliary adjustment chamber 18Ab.

The first auxiliary adjustment chamber 18Ab is provided as a space for further carrying out adjustment of the oxygen partial pressure by a first auxiliary pump cell 56A, with respect to the gas to be measured which is introduced through the fourth diffusion rate control member 40A, after the oxygen concentration (oxygen partial pressure) has been adjusted beforehand in the first main adjustment chamber 18Aa. In accordance with this feature, the oxygen concentration inside the first auxiliary adjustment chamber 18Ab can be kept constant with high accuracy, and therefore, the first sensor cell 15A is made capable of measuring the NOx concentration with high accuracy.

The first auxiliary pump cell 56A is an electrochemical pump cell, and is constituted by a first auxiliary pump electrode 58A, which is provided substantially over the entirety of the lower surface of the second solid electrolyte layer 32 facing toward the first auxiliary adjustment chamber 18Ab, the common exterior side pump electrode 46, and the second solid electrolyte layer 32.

Moreover, in the same manner as the first main interior side pump electrode 44A, the first auxiliary pump electrode 58A is also formed using a material that weakens the reduction capability with respect to the NOx component within the gas to be measured.

The first auxiliary pump cell 56A, by applying a desired second voltage Vp2 between the first auxiliary pump electrode 58A and the exterior side pump electrode 46, is capable of pumping out oxygen within the atmosphere inside the first auxiliary adjustment chamber 18Ab into the external space, or alternatively, is capable of pumping in oxygen from the external space into the first auxiliary adjustment chamber 18Ab.

Further, in order to control the oxygen partial pressure within the atmosphere inside the first auxiliary adjustment chamber 18Ab, an electrochemical sensor cell, and more specifically, a second oxygen partial pressure detecting sensor cell 50B for controlling the first auxiliary pump, is constituted by the first auxiliary pump electrode 58A, the reference electrode 52, the second solid electrolyte layer 32, the spacer layer 30, and the first solid electrolyte layer 28.

Moreover, the first auxiliary pump cell 56A carries out pumping by a second variable power source 48B, the voltage of which is controlled based on a second electromotive force V2 detected by the second oxygen partial pressure detecting sensor cell 50B. Consequently, the oxygen partial pressure within the atmosphere inside the first auxiliary adjustment chamber 18Ab is controlled so as to become a low partial pressure that does not substantially influence the measurement of NOx.

Further, together therewith, a second pump current value Ip2 of the first auxiliary pump cell 56A is used so as to control the second electromotive force V2 of the second oxygen partial pressure detecting sensor cell 50B. More specifically, the second pump current Ip2 is input as a control signal to the second oxygen partial pressure detecting sensor cell 50B, and by controlling the second electromotive force V2, the gradient of the oxygen partial pressure within the gas to be measured, which is introduced through the fourth diffusion rate control member 40A into the first auxiliary adjustment chamber 18Ab, is controlled so as to remain constant at all times. Furthermore, if the first variable power source 48A of the first main pump cell 42A is feedback-controlled, in a manner so that the second pump current value Ip2 becomes constant, the accuracy of the oxygen partial pressure control within the first auxiliary adjustment chamber 18Ab is further improved. When the first sensor cell 15A is used as a NOx sensor, by the actions of the first main pump cell 42A and the first auxiliary pump cell 56A, the oxygen concentration inside the first auxiliary adjustment chamber 18Ab is maintained at a predetermined value with high accuracy for each of the respective conditions.

The third diffusion rate control member 38A imparts a predetermined diffusion resistance to the gas to be measured, the oxygen concentration (oxygen partial pressure) of which is controlled by operation of the first auxiliary pump cell 56A in the first auxiliary adjustment chamber 18Ab, and is a location that guides the gas to be measured into the first measurement chamber 20A.

In the first sensor cell 15A, measurement of the NOx concentration is primarily performed by operation of a first measurement pump cell 60A provided in the first measurement chamber 20A. As shown in FIG. 2, the first measurement pump cell 60A is an electrochemical pump cell constituted by a first measurement electrode 62A, the common exterior side pump electrode 46, the second solid electrolyte layer 32, the spacer layer 30, and the first solid electrolyte layer 28. The first measurement electrode 62A is provided, for example, directly on the upper surface of the first solid electrolyte layer 28 inside the first measurement chamber 20A, and is a porous cermet electrode made of a material whose reduction capability with respect to the NOx component within the gas to be measured is higher than that of the first main interior side pump electrode 44A. The first measurement electrode 62A also functions as a NOx reduction catalyst for reducing NOx existing within the atmosphere above the first measurement electrode 62A.

The first measurement pump cell 60A is capable of pumping out oxygen that is generated by the decomposition of nitrogen oxide within the atmosphere around the periphery of the first measurement electrode 62A (inside the first measurement chamber 20A), and can detect the generated amount as a first pump current value Ip3, and more specifically, as a sensor output (a first measurement pump current value Ip3) of the first sensor cell 15A.

Further, in order to detect the oxygen partial pressure around the periphery of the first measurement electrode 62A (inside the first measurement chamber 20A), an electrochemical sensor cell, and more specifically, a third oxygen partial pressure detecting sensor cell 50C for controlling the measurement pump, is constituted by the first solid electrolyte layer 28, the spacer layer 30, the first measurement electrode 62A, and the reference electrode 52. A third variable power source 48C is controlled based on a third electromotive force V3 detected by the third oxygen partial pressure detecting sensor cell 50C.

The gas to be measured, which is introduced into the first auxiliary adjustment chamber 18Ab, reaches the first measurement electrode 62A inside the first measurement chamber 20A through the third diffusion rate control member 38A, under a condition in which the oxygen partial pressure is controlled. Nitrogen oxide existing within the gas to be measured around the periphery of the first measurement electrode 62A is reduced to thereby generate oxygen. Then, the generated oxygen is subjected to pumping by the first measurement pump cell 60A. At this time, a third voltage Vp3 of the third variable power source 48C is controlled in a manner so that the third electromotive force V3 detected by the third oxygen partial pressure detecting sensor cell 50C becomes constant. The amount of oxygen generated around the periphery of the first measurement electrode 62A is proportional to the concentration of nitrogen oxide within the gas to be measured. Accordingly, the nitrogen oxide concentration within the gas to be measured can be calculated using the first measurement pump current value Ip3 of the first measurement pump cell 60A. More specifically, the first measurement pump cell 60A measures the concentration of a specified component (NO) within the first measurement chamber 20A.

Furthermore, in the first sensor cell 15A, a first heater 72A is formed in a manner of being sandwiched from above and below between the second substrate layer 26b and the third substrate layer 26c. The first heater 72A generates heat by being supplied with power from the exterior through a non-illustrated heater electrode provided on a lower surface of the first substrate layer 26a. As a result of the heat generated by the first heater 72A, the oxygen ion conductivity of the solid electrolyte that constitutes the first sensor cell 15A is enhanced. The first heater 72A is embedded over the entire region of the first preliminary adjustment chamber 22A and the first oxygen concentration adjustment chamber 18A, and the first measurement chamber 20A, whereby a predetermined location of the first sensor cell 15A can be heated and maintained at a predetermined temperature. Moreover, a first heater insulating layer 74A made of alumina or the like is formed on the upper and lower surfaces of the first heater 72A, for the purpose of obtaining electrical insulation thereof from the second substrate layer 26b and the third substrate layer 26c.

Further, the first sensor cell 15A includes a first switch SW1 which controls operation of a first preliminary adjustment pump cell 80A, which will be described later, so as to be turned ON or OFF. The first preliminary adjustment chamber 22A functions as a space for the purpose of adjusting an oxygen partial pressure within the gas to be measured that is introduced from the first gas introduction port 16A. The oxygen partial pressure is adjusted by operation of the first preliminary adjustment pump cell 80A.

The first preliminary adjustment pump cell 80A is a preliminary electrochemical pump cell that is operated when the first switch SW1 is turned ON. The first preliminary adjustment pump cell 80A is constituted by a first preliminary pump electrode 82A, which is provided substantially over the entirety of the lower surface of the second solid electrolyte layer 32 facing toward the first preliminary adjustment chamber 22A, the exterior side pump electrode 46, and the second solid electrolyte layer 32.

Moreover, in the same manner as the first main interior side pump electrode 44A, the first preliminary pump electrode 82A is also formed using a material that weakens the reduction capability with respect to the NOx component within the gas to be measured. More specifically, for example, components of both Pt and Au are contained therein, wherein the composition ratio Au/(Pt+Au) is greater than or equal to 4% and less than or equal to 20%. These components make up a porous cermet.

The first preliminary adjustment pump cell 80A, by applying a desired first preliminary voltage Vpa between the first preliminary pump electrode 82A and the exterior side pump electrode 46, is capable of pumping out oxygen within the atmosphere inside the first preliminary adjustment chamber 22A into the external space, or alternatively, is capable of pumping in oxygen from the external space into the first preliminary adjustment chamber 22A.

Further, the first sensor cell 15A includes a first preliminary oxygen partial pressure detecting sensor cell 84A for controlling the first preliminary pump, in order to control the oxygen partial pressure within the atmosphere inside the first preliminary adjustment chamber 22A. The first preliminary oxygen partial pressure detecting sensor cell 84A includes the first preliminary pump electrode 82A, the reference electrode 52, the second solid electrolyte layer 32, the spacer layer 30, and the first solid electrolyte layer 28.

Moreover, the first preliminary adjustment pump cell 80A performs pumping at a first preliminary variable power supply 86A, whose voltage is controlled based on the first preliminary electromotive force Va detected by the first preliminary oxygen partial pressure detecting sensor cell 84A. Consequently, the oxygen partial pressure within the atmosphere inside the first preliminary adjustment chamber 22A is controlled so as to become a low partial pressure that does not substantially influence the measurement of NOx.

Further, together therewith, a first preliminary pump current value Ipa thereof is used so as to control the electromotive force of the first preliminary oxygen partial pressure detecting sensor cell 84A. More specifically, the first preliminary pump current Ipa is input as a control signal to the first preliminary oxygen partial pressure detecting sensor cell 84A, and by controlling the first preliminary electromotive force Va, the gradient of the oxygen partial pressure within the gas to be measured, which is introduced from the first diffusion rate control member 34A into the first preliminary adjustment chamber 22A, is controlled so as to remain constant at all times.

The first preliminary adjustment chamber 22A also functions as a buffer space. More specifically, it is possible to cancel fluctuations in the concentration of the gas to be measured, which are caused by pressure fluctuations of the gas to be measured in the external space (pulsations in the exhaust pressure, in the case that the gas to be measured is an exhaust gas of an automobile).

On the other hand, as shown in FIG. 3, the second sensor cell 15B has a similar configuration to that of the aforementioned first sensor cell 15A, and includes the second main pump cell 42B, a second auxiliary pump cell 56B, a fourth oxygen partial pressure detecting sensor cell 50D, a fifth oxygen partial pressure detecting sensor cell 50E, and a sixth oxygen partial pressure detecting sensor cell 50F.

The second main pump cell 42B, in the same manner as the first main pump cell 42A, comprises a second electrochemical pump cell (main electrochemical pumping cell), which is constituted by including the second main interior side pump electrode 44B, the common exterior side pump electrode 46, and an oxygen ion conductive solid electrolyte which is sandwiched between the two pump electrodes.

By applying a fourth pump voltage Vp4 supplied from a fourth variable power source 48D for the second sensor cell 15B, and by allowing a fourth pump current Ip4 to flow between the common exterior side pump electrode 46 and the second main interior side pump electrode 44B, it is possible to pump oxygen in the interior of the second main adjustment chamber 18Ba into the external space, or alternatively, to pump oxygen in the external space into the second main adjustment chamber 18Ba.

The second auxiliary pump cell 56B is an electrochemical pump cell, and in the same manner as the aforementioned first auxiliary pump cell 56A, is constituted by a second auxiliary pump electrode 58B, which is provided substantially over the entirety of the lower surface of the second solid electrolyte layer 32 facing toward the second auxiliary adjustment chamber 18Bb, the common exterior side pump electrode 46, and the second solid electrolyte layer 32.

The second auxiliary pump cell 56B, by applying a desired fifth voltage Vp5 between the second auxiliary pump electrode 58B and the exterior side pump electrode 46, is capable of pumping out oxygen within the atmosphere inside the second auxiliary adjustment chamber 18Bb into the external space, or alternatively, is capable of pumping in oxygen from the external space into the second auxiliary adjustment chamber 18Bb.

The fourth oxygen partial pressure detecting sensor cell 50D, in the same manner as the first oxygen partial pressure detecting sensor cell 50A, is constituted by the second main interior side pump electrode 44B, the common reference electrode 52 sandwiched between the first solid electrolyte layer 28 and an upper surface of the third substrate layer 26c, and an oxygen ion conductive solid electrolyte sandwiched between these electrodes.

The fourth oxygen partial pressure detecting sensor cell 50D generates a fourth electromotive force V4 between the second main interior side pump electrode 44B and the reference electrode 52, which is caused by a difference in oxygen concentration between the atmosphere inside the second main adjustment chamber 18Ba and the reference gas in the reference gas introduction space 41.

The fourth electromotive force V4 generated in the fourth oxygen partial pressure detecting sensor cell 50D changes depending on the oxygen partial pressure of the atmosphere existing in the second main adjustment chamber 18Ba. In accordance with the aforementioned fourth electromotive force V4, the second sensor cell 15B feedback-controls the fourth variable power source 48D of the second main pump cell 42B. Consequently, the fourth pump voltage Vp4, which is applied by the fourth variable power source 48D to the second main pump cell 42B, can be controlled in accordance with the oxygen partial pressure of the atmosphere in the second main adjustment chamber 18Ba.

Further, in order to control the oxygen partial pressure within the atmosphere inside the second auxiliary adjustment chamber 18Bb, an electrochemical sensor cell, and more specifically, the fifth oxygen partial pressure detecting sensor cell 50E for controlling the second auxiliary pump, is constituted by the second auxiliary pump electrode 58B, the reference electrode 52, the second solid electrolyte layer 32, the spacer layer 30, and the first solid electrolyte layer 28.

The second auxiliary pump cell 56B carries out pumping by a fifth variable power source 48E, the voltage of which is controlled based on a fifth electromotive force V5 detected by the fifth oxygen partial pressure detecting sensor cell 50E. Consequently, the oxygen partial pressure within the atmosphere inside the second auxiliary adjustment chamber 18Bb is controlled so as to become a low partial pressure that does not substantially influence the measurement of NOx.

Further, together therewith, a fifth pump current value Ip5 of the second auxiliary pump cell 56B is used so as to control the fifth electromotive force V5 of the fifth oxygen partial pressure detecting sensor cell 50E. Stated otherwise, the gradient of the oxygen partial pressure within the gas to be measured, which is introduced into the second auxiliary adjustment chamber 18Bb, is controlled so as to remain constant at all times.

Further, in order to detect the oxygen partial pressure around the periphery of a second measurement electrode 62B (inside the second measurement chamber 20B), an electrochemical sensor cell, and more specifically, the sixth oxygen partial pressure detecting sensor cell 50F for controlling the measurement pump, is constituted by the first solid electrolyte layer 28, the spacer layer 30, the second measurement electrode 62B, and the reference electrode 52. A sixth variable power source 48F is controlled based on a sixth electromotive force V6 detected by the sixth oxygen partial pressure detecting sensor cell 50F.

The gas to be measured, which is introduced into the second auxiliary adjustment chamber 18Bb, reaches the second measurement electrode 62B inside the second measurement chamber 20B through the third diffusion rate control member 38B, under a condition in which the oxygen partial pressure is controlled. Nitrogen oxide existing within the gas to be measured around the periphery of the second measurement electrode 62B is reduced to thereby generate oxygen. Then, the generated oxygen is subjected to pumping by a second measurement pump cell 60B. At this time, a sixth voltage Vp6 of the sixth variable power source 48F is controlled in a manner so that the sixth electromotive force V6 detected by the sixth oxygen partial pressure detecting sensor cell 50F becomes constant. The amount of oxygen generated around the periphery of the second measurement electrode 62B is proportional to the concentration of nitrogen oxide within the gas to be measured. Accordingly, the nitrogen oxide concentration within the gas to be measured can be calculated using the second measurement pump current value Ip6 of the second measurement pump cell 60B. More specifically, the second measurement pump cell 60B measures the concentration of a specified component (NO) within the second measurement chamber 20B.

Further, the second sensor cell 15B includes an electrochemical oxygen detecting cell 70. The oxygen detecting cell 70 includes the second solid electrolyte layer 32, the spacer layer 30, the first solid electrolyte layer 28, the third substrate layer 26c, the exterior side pump electrode 46, and the reference electrode 52. In accordance with the electromotive force Vr obtained by the oxygen detecting cell 70, it is possible to detect the oxygen partial pressure within the gas to be measured existing externally of the sensor element 12.

Further, in the second sensor cell 15B, a second heater 72B is formed similarly to the aforementioned first heater 72A, in a manner of being sandwiched from above and below between the second substrate layer 26b and the third substrate layer 26c. The second heater 72B is embedded over the entire region of the second preliminary adjustment chamber 22B and the second oxygen concentration adjustment chamber 18B, and the second measurement chamber 20B, whereby a predetermined location of the second sensor cell 15B can be heated and maintained at a predetermined temperature. Moreover, a second heater insulating layer 74B made of alumina or the like is formed on the upper and lower surfaces of the second heater 72B, for the purpose of obtaining electrical insulation thereof from the second substrate layer 26b and the third substrate layer 26c. The first heater 72A and the second heater 72B may be configured by one common heater, and in such a case, the first heater insulating layer 74A and the second heater insulating layer 74B are also provided in common.

Further, in the second sensor cell 15B as well, as shown in FIG. 3, there is included a second switch SW2 which controls operation of a second preliminary adjustment pump cell 80B, which will be described later, so as to be turned ON or OFF. The second preliminary adjustment chamber 22B is provided as a space for the purpose of adjusting an oxygen partial pressure within the gas to be measured that is introduced from the second gas introduction port 16B. The oxygen partial pressure is adjusted by operation of the second preliminary adjustment pump cell 80B.

The second preliminary adjustment pump cell 80B is a preliminary electrochemical pump cell that is operated when the second switch SW2 is turned ON. The second preliminary adjustment pump cell 80B is a preliminary electrochemical pump cell, and is constituted by a second preliminary pump electrode 82B, which is provided substantially over the entirety of the lower surface of the second solid electrolyte layer 32 facing toward the second preliminary adjustment chamber 22B, the exterior side pump electrode 46, and the second solid electrolyte layer 32.

Moreover, in the same manner as the first preliminary pump electrode 82A (see FIG. 2), the second preliminary pump electrode 82B is also formed using a material that weakens the reduction capability with respect to the NOx component within the gas to be measured. More specifically, for example, components of both Pt and Au are contained therein, wherein the composition ratio Au/(Pt+Au) is greater than or equal to 4% and less than or equal to 20%. These components make up a porous cermet.

The second preliminary adjustment pump cell 80B, by applying a desired second preliminary voltage Vpb between the second preliminary pump electrode 82B and the exterior side pump electrode 46, is capable of pumping out oxygen within the atmosphere inside the second preliminary adjustment chamber 22B into the external space, or alternatively, is capable of pumping in oxygen from the external space into the second preliminary adjustment chamber 22B.

Further, the second sensor cell 15B includes a second preliminary oxygen partial pressure detecting sensor cell 84B for controlling the second preliminary pump, in order to control the oxygen partial pressure within the atmosphere inside the second preliminary adjustment chamber 22B. The second preliminary oxygen partial pressure detecting sensor cell 84B includes the second preliminary pump electrode 82B, the reference electrode 52, the second solid electrolyte layer 32, the spacer layer 30, and the first solid electrolyte layer 28.

Moreover, the second preliminary adjustment pump cell 80B performs pumping at a second preliminary variable power supply 86B, whose voltage is controlled based on the second preliminary electromotive force Vb detected by the second preliminary oxygen partial pressure detecting sensor cell 84B. Consequently, the oxygen partial pressure within the atmosphere inside the second preliminary adjustment chamber 22B is controlled so as to become a low partial pressure that does not substantially influence the measurement of NOx.

Further, together therewith, a second preliminary pump current value Ipb thereof is used so as to control the electromotive force of the second preliminary oxygen partial pressure detecting sensor cell 84B. More specifically, the second preliminary pump current Ipb is input as a control signal to the second preliminary oxygen partial pressure detecting sensor cell 84B, and by controlling the second preliminary electromotive force Vb, the gradient of the oxygen partial pressure within the gas to be measured, which is introduced from the first diffusion rate control member 34B into the second preliminary adjustment chamber 22B, is controlled so as to remain constant at all times.

The second preliminary adjustment chamber 22B also functions as a buffer space. More specifically, it is possible to cancel fluctuations in the concentration of the gas to be measured, which are caused by pressure fluctuations of the gas to be measured in the external space (pulsations in the exhaust pressure, in the case that the gas to be measured is an exhaust gas of an automobile).

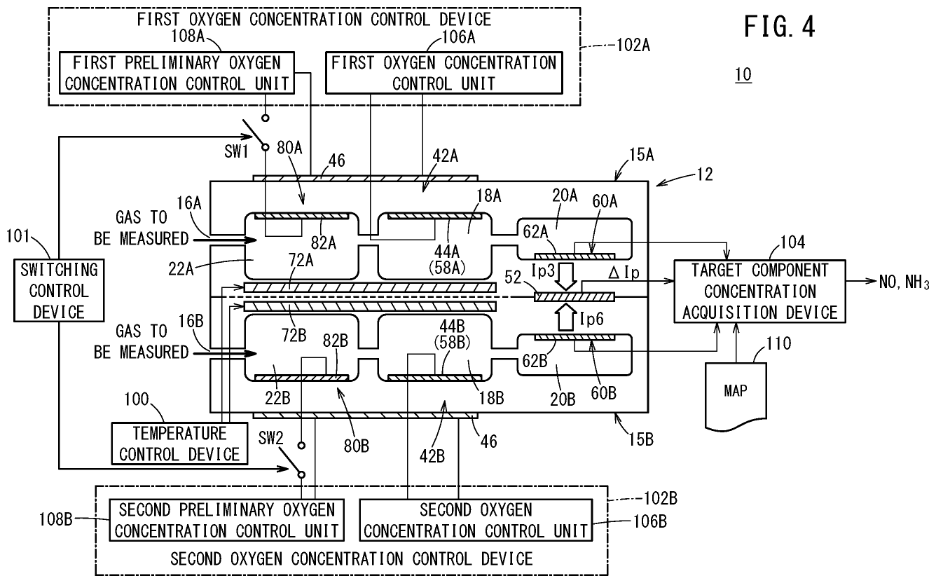

Furthermore, as shown schematically in FIG. 4, the gas sensor 10 includes a temperature control device 100, a switching control device 101, a first oxygen concentration control device 102A, a second oxygen concentration control device 102B, and a target component concentration acquisition device 104.

The temperature control device 100 controls the supply of current to the first heater 72A and the second heater 72B of the sensor element 12, and thereby controls the temperature of the first sensor cell 15A and the second sensor cell 15B.

The switching control device 101 performs a switching control for the first switch SW1 and the second switch SW2. For example, in the case of operating the first preliminary adjustment pump cell 80A, the first switch SW1 is turned ON and the second switch SW2 is turned OFF. Conversely, in the case of operating the second preliminary adjustment pump cell 80B, the first switch SW1 is turned OFF and the second switch SW2 is turned ON.

The first oxygen concentration control device 102A includes a first oxygen concentration control unit 106A that controls the oxygen concentration inside the first oxygen concentration adjustment chamber 18A of the first sensor cell 15A, and a first preliminary oxygen concentration control unit 108A that controls the oxygen concentration inside the first preliminary adjustment chamber 22A of the first sensor cell 15A.

The second oxygen concentration control device 102B includes a second oxygen concentration control unit 106B that controls the oxygen concentration inside the second oxygen concentration adjustment chamber 18B of the second sensor cell 15B, and a second preliminary oxygen concentration control unit 108B that controls the oxygen concentration inside the second preliminary adjustment chamber 22B of the second sensor cell 15B.

The target component concentration acquisition device 104 acquires the concentrations of the first target component (NO) and the second target component (NH₃), on the basis of the difference (amount of change ΔIp) between the first measurement pump current value Ip3 flowing to the first measurement pump cell 60A of the first sensor cell 15A and the second measurement pump current value Ip6 flowing to the second measurement pump cell 60B of the second sensor cell 15B, the second measurement pump current value Ip6 (the total concentration), and a later-described map 110.

Moreover, the temperature control device 100, the switching control device 101, the first oxygen concentration control device 102A, the second oxygen concentration control device 102B, and the target component concentration acquisition device 104 are constituted by one or more processors having, for example, one or a plurality of CPUs (central processing units), memory devices, and the like. The one or more processors are software-based functional units in which predetermined functions are realized, for example, by the CPUs executing programs stored in a storage device. Of course, the processors may be constituted by an integrated circuit such as an FPGA (Field-Programmable Gate Array), in which the plurality of processors are connected according to the functions thereof. Moreover, as noted above, the map 110 may be stored in advance in the storage device, which is one of the peripheral circuits of the gas sensor. Of course, the map 110, which is acquired (stored in the above-described storage device) through the communication means, may also be used.

With a NOx sensor possessed by a conventional serial-arranged two-chamber type structure, with respect to the target components of NO and NH₃, after the totality thereof has been converted into NO inside the oxygen concentration adjustment chamber, the converted components are introduced into the measurement chamber, and then the total amount of these two components is measured. Stated otherwise, it has been impossible to measure the concentrations of each of the two target components, that is, the respective concentrations of NO and NH₃.

In contrast thereto, the gas sensor 10 is equipped with the aforementioned first sensor cell 15A, the second sensor cell 15B, the temperature control device 100, the switching control device 101, the first oxygen concentration control device 102A, the second oxygen concentration control device 102B, and the target component concentration acquisition device 104, whereby the respective concentrations of NO and NH₃ can be acquired.

The temperature control device 100 feedback-controls the first heater 72A and the second heater 72B on the basis of a preset sensor temperature condition, and the measured value from a temperature sensor (not shown) that measures the temperature of the sensor element 12, whereby the temperature of the sensor element 12 is adjusted to a temperature in accordance with the aforementioned condition.

On the basis of the preset oxygen concentration condition inside the first oxygen concentration adjustment chamber 18A, and the first electromotive force V1 generated in the first oxygen partial pressure detecting sensor cell 50A (see FIG. 2), the first oxygen concentration control unit 106A of the first oxygen concentration control device 102A feedback-controls the first variable power source 48A, thereby adjusting the oxygen concentration inside the first oxygen concentration adjustment chamber 18A to a concentration in accordance with the aforementioned condition.

On the basis of the preset oxygen concentration condition inside the second oxygen concentration adjustment chamber 18B, and the fourth electromotive force V4 generated in the fourth oxygen partial pressure detecting sensor cell 50D (see FIG. 3), the second oxygen concentration control unit 106B of the second oxygen concentration control device 102B feedback-controls the fourth variable power source 48D, thereby adjusting the oxygen concentration inside the second oxygen concentration adjustment chamber 18B to a concentration in accordance with the aforementioned condition.

In this manner, by the first oxygen concentration control device 102A and the second oxygen concentration control device 102B or the temperature control device 100, or alternatively, by the first oxygen concentration control device 102A, the second oxygen concentration control device 102B, and the temperature control device 100, the gas sensor 10 performs a control so as to convert NH₃ into NO at a ratio suitable for measurement of NH₃, without causing decomposition of NO inside the first oxygen concentration adjustment chamber 18A and the second oxygen concentration adjustment chamber 18B.

On the basis of the preset oxygen concentration condition, and the first preliminary electromotive force Va generated in the first preliminary oxygen partial pressure detecting sensor cell 84A (see FIG. 2), the first preliminary oxygen concentration control unit 108A of the first oxygen concentration control device 102A feedback-controls the first preliminary variable power source 86A, thereby adjusting the oxygen concentration inside the first preliminary adjustment chamber 22A to a concentration in accordance with the condition. By the first preliminary oxygen concentration control unit 108A, NH₃ is converted into NO at a ratio suitable for measurement of NH₃, without causing decomposition of NO inside the first preliminary adjustment chamber 22A in the first sensor cell 15A.

Similarly, on the basis of the preset oxygen concentration condition, and the second preliminary electromotive force Vb generated in the second preliminary oxygen partial pressure detecting sensor cell 84B (see FIG. 3), the second preliminary oxygen concentration control unit 108B of the second oxygen concentration control device 102B feedback-controls the second preliminary variable power source 86B, thereby adjusting the oxygen concentration inside the second preliminary adjustment chamber 22B to a concentration in accordance with the condition. By the second preliminary oxygen concentration control unit 108B, NH₃ is converted into NO at a ratio capable of being used for measurement of NH₃, without causing decomposition of NO inside the second preliminary adjustment chamber 22B in the second sensor cell 15B.

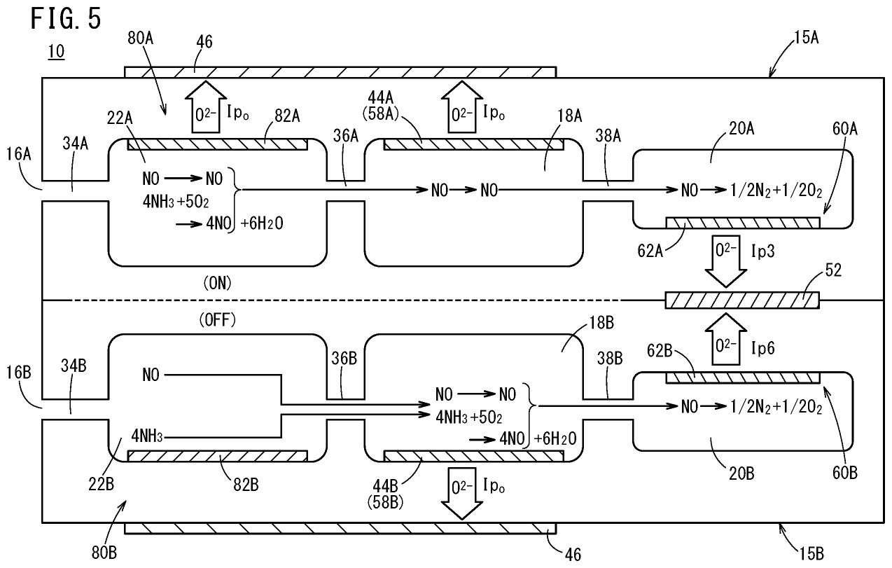

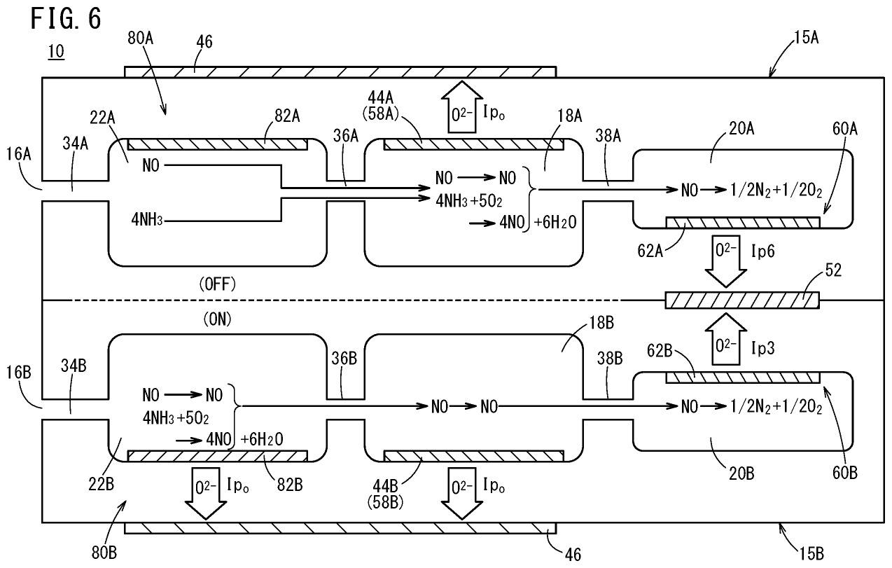

Next, processing operations of the gas sensor 10 will be described with reference also to FIGS. 5 and 6.

First, in the first sensor cell 15A, as shown in FIG. 5, since the first preliminary adjustment pump cell 80A is turned ON, the NH₃ introduced into the first preliminary adjustment chamber 22A through the first gas introduction port 16A is subjected to an oxidation reaction of NH₃→NO inside the first preliminary adjustment chamber 22A, whereupon all of the NH₃ introduced through the first gas introduction port 16A is converted into NO. Accordingly, although the NH₃ passes through the first diffusion rate control member 34A at the NH₃ diffusion coefficient of 2.2 cm²/sec, after having passed through the second diffusion rate control member 36A on the innermost side from the first preliminary adjustment chamber 22A, movement into the first measurement chamber 20A occurs at the NO diffusion coefficient of 1.8 cm²/sec.

On the other hand, in the second sensor cell 15B, since the second preliminary adjustment pump cell 80B is in a state of being turned OFF, the NH₃ that was introduced through the second gas introduction port 16B reaches the second oxygen concentration adjustment chamber 18B. In the second oxygen concentration adjustment chamber 18B, by operation of the second oxygen concentration control device 102B (see FIG. 4), a control is performed so as to convert all of the NH₃ into NO, and therefore, the NH₃ that has flowed into the second oxygen concentration adjustment chamber 18B causes an oxidation reaction of NH₃→NO to occur inside the second oxygen concentration adjustment chamber 18B, and all of the NH₃ inside the second oxygen concentration adjustment chamber 18B is converted into NO. Accordingly, the NH₃ that was introduced through the second gas introduction port 16B passes through the first diffusion rate control member 34B and the second diffusion rate control member 36B at the NH₃ diffusion coefficient of 2.2 cm²/sec, and after being converted into NO inside the second oxygen concentration adjustment chamber 18B, passes through the third diffusion rate control member 38B at the NO diffusion coefficient of 1.8 cm²/sec, and moves into the adjacent second measurement chamber 20B.

More specifically, in the first sensor cell 15A, the location where the oxidation reaction of NH₃ takes place is the first preliminary adjustment chamber 22A, and in the second sensor cell 15B, the location where the oxidation reaction of NH₃ takes place is the second oxygen concentration adjustment chamber 18B. Since NO and NH₃ each possess different diffusion coefficients, the difference between passing through the second diffusion rate control members (36A and 36B) with NO or passing therethrough with NH₃ corresponds to a difference in the amount of NO that flows into the first measurement chamber 20A and the second measurement chamber 20B. Such a feature brings about a difference between the first measurement pump current value Ip3 of the first measurement pump cell 60A, and the second measurement pump current value Ip6 of the second measurement pump cell 60B. However, significantly, the second measurement pump current value Ip6 of the second measurement pump cell 60B corresponds to the total value of the NH₃ concentration and the NO concentration within the measurement gas.

Additionally, the amount of change ΔIp between the first measurement pump current value Ip3 and the second measurement pump current value Ip6 changes according to the NH₃ concentration within the gas to be measured. Therefore, the respective concentrations of NO and NH₃ can be obtained from the second measurement pump current value Ip6 (the total concentration of NO and NH₃) that flows to the second measurement pump cell 60B, and the aforementioned amount of change ΔIp (the NH₃ concentration).

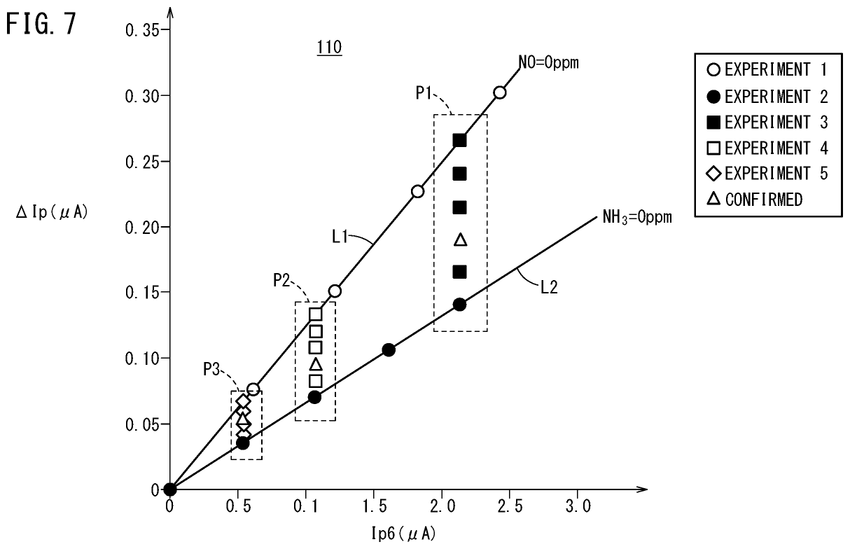

Accordingly, with the target component concentration acquisition device 104 (see FIG. 4), the respective concentrations of NO and NH₃ can be acquired on the basis of the amount of change ΔIp between the first measurement pump current value Ip3 and the second measurement pump current value Ip6, the second measurement pump current value Ip6, and for example, the map 110 (see FIG. 7).

When the map 110 is shown graphically, as shown in FIG. 7, a graph is produced in which the second measurement pump current value Ip6 (μA) is set on the horizontal axis, and the amount of change ΔIp (μA) between the first measurement pump current value Ip3 and the second measurement pump current value Ip6 is set on the vertical axis. In FIG. 7, there are shown representatively a first characteristic line L1 and a second characteristic line L2, and a first plot group P1, a second plot group P2, and a third plot group P3 of the amount of change ΔIp, in which the NO concentration conversion values thereof pertain to a 100 ppm system, a 50 ppm system, and a 25 ppm system.

The first characteristic line L1 shows a characteristic, in relation to a case in which the NO concentration conversion value is 0 ppm, i.e., a case in which NO is not contained in the gas to be measured, for cases in which the NH₃ concentration conversion value is changed between 0 ppm, 25 ppm, 50 ppm, 75 ppm, and 100 ppm.

The second characteristic line L2 shows a characteristic, in relation to a case in which the NH₃ concentration conversion value is 0 ppm, i.e., a case in which NH₃ is not contained in the gas to be measured, for cases in which the NO concentration conversion value is changed between 0 ppm, 25 ppm, 50 ppm, 75 ppm, and 100 ppm.

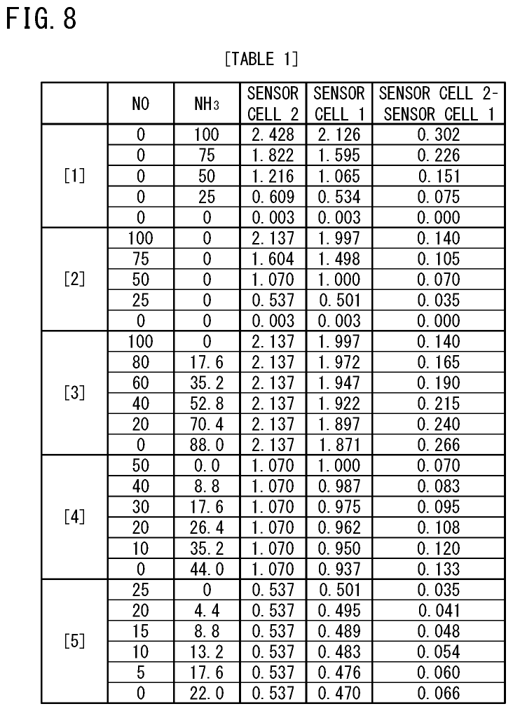

When the graph of FIG. 7 is shown in the form of a table to facilitate understanding, the contents thereof are as shown in FIG. 8. The contents thereof can be determined, for example, by carrying out Experiments 1 to 5, which will be described later.

In the table of FIG. 8, the contents presented in the first section [1] correspond to the first characteristic line L1 of FIG. 7, and the contents presented in the second section [2] correspond to the second characteristic line L2 of FIG. 7. From a comparison of sections [1] and [2], it can be understood that NH₃ possesses a sensitivity that is 1.14 times that of NO. Such a feature is manifested on the basis of the difference in the diffusion coefficients of NH₃ and NO, and is determined by the temperature of the sensor element 12 and the oxygen concentration within the internal space. Further, in the table of FIG. 8, the contents of the third section [3] correspond to the first plot group P1 of FIG. 7, the contents of the fourth section [4] correspond to the second plot group P2 of FIG. 7, and the contents of the fifth section [5] correspond to the third plot group P3 of FIG. 7.

In addition, referring to the contents of the third section [3], the fourth section [4], and the fifth section [5] within Table 1 of FIG. 8, the NO concentration is acquired by calculating the total concentration (the NO conversion value) based on the second measurement pump current value Ip6, and more specifically, any one of the 100 ppm system, the 50 ppm system, and the 25 ppm system, acquiring the NH₃ concentration on the basis of the amount of change ΔIp, and subtracting the NH₃ concentration from the total concentration.

For example, in the case that the second measurement pump current value Ip6 is 0.537 (μA), the fact that the total concentration is 25 ppm is calculated from the fifth section [5] of Table 1 of FIG. 8. In addition, in the case that the amount of change ΔIp is 0.041 (μA), from the fifth section [5] of Table 1 of FIG. 8, the NH₃ concentration is 4.4 ppm. Accordingly, taking into consideration the difference in sensitivity between NH₃ and NO, the NO concentration is 25−4.4×1.14=approximately 20.0 ppm.

Moreover, in the case that no corresponding amount of change ΔIp exists on the map 110, the amount of change ΔIp that is closest thereto on the map 110 may be specified to thereby calculate the total concentration, and together therewith, the NH₃ concentration may be determined, for example, by a known approximation calculation. In addition, the NO concentration may be determined by subtracting the determined NH₃ concentration from the calculated total concentration. Alternatively, the concentration of NH₃ which is the second target component may be calculated on the basis of a correlation equation between the respective concentrations of NH₃ and NO, ΔIp, and Ip6, and the concentration of NO which is the first target component may be calculated by subtracting the concentration of the second target component from the total concentration.

Next, a description will be given concerning an experimental example for the purpose of obtaining the map 110.

(1) The above-described sensor element 12 is manufactured, and the metal components are assembled into a sensor shape and attached to a model gas measurement apparatus. In addition, by the first heater 72A and the second heater 72B being incorporated into the sensor element 12, the sensor element 12 is heated to approximately 800° C.

(2) The voltage applied between the first main interior side pump electrode 44A and the exterior side pump electrode 46, as well as the voltage applied between the second main interior side pump electrode 44B and the exterior side pump electrode 46 are feedback-controlled, in a manner so that the electromotive force between the first main interior side pump electrode 44A of the first sensor cell 15A and the reference electrode 52, and the electromotive force between the second main interior side pump electrode 44B of the second sensor cell 15B and the reference electrode 52 become 230 mV.

(3) Next, the voltage applied between the first main interior side pump electrode 44A and the exterior side pump electrode 46, as well as the voltage applied between the second main interior side pump electrode 44B and the exterior side pump electrode 46 are feedback-controlled, in a manner so that the electromotive force between the first auxiliary pump electrode 58A of the first sensor cell 15A and the reference electrode 52, and the electromotive force between the second auxiliary pump electrode 58B of the second sensor cell 15B and the reference electrode 52 become 385 mV.

(4) Furthermore, the voltage applied between the first measurement electrode 62A and the exterior side pump electrode 46, as well as the voltage applied between the second measurement electrode 62B and the exterior side pump electrode 46 are feedback-controlled, in a manner so that the electromotive force between the first measurement electrode 62A of the first measurement pump cell 60A and the reference electrode 52 in the first sensor cell 15A, and the electromotive force between the second measurement electrode 62B of the second measurement pump cell 60B and the reference electrode 52 in the second sensor cell 15B become 400 mV.

(5) In a state in which the first switch SW1 is turned ON, and the first preliminary adjustment pump cell 80A of the first sensor cell 15A is turned ON, a state is brought about in which the second switch SW2 is turned OFF, and the second preliminary adjustment pump cell 80B of the second sensor cell 15B is turned OFF. Thereafter, the voltage applied between the first preliminary pump electrode 82A and the exterior side pump electrode 46 was feedback-controlled, in a manner so that the electromotive force between the first preliminary pump electrode 82A and the reference electrode 52 of the first preliminary adjustment pump cell 80A became a predetermined voltage.

(6) Next, N2 and 3% of H₂O were made to flow as a base gas at 120 L/min to the model gas measurement apparatus, and upon having measured the current flowing to the first measurement pump cell 60A and the second measurement pump cell 60B, an offset current flowing to the first measurement pump cell 60A and the second measurement pump cell 60B was determined to be 0.003 μA.

(7) Next, while N2 and 3% of H₂O continued to flow as a base gas at 120 L/min to the model gas measurement apparatus, and while maintaining a total gas flow rate of 120 L/min, by the addition of NH₃ at amounts of 25, 50, 75, and 100 ppm, the first measurement pump current Ip3 and the second measurement pump current Ip6 flowing to the first measurement pump cell 60A and the second measurement pump cell 60B were measured (Experiment 1: refer to the first characteristic line L1 of FIG. 7, and the first section [1] of Table 1 of FIG. 8).

(8) Next, while N2 and 3% of H₂O continued to flow as a base gas at 120 L/min to the model gas measurement apparatus, and while maintaining a total gas flow rate of 120 L/min, by a stepwise addition of NO at amounts of 25, 50, 75, and 100 ppm, the first measurement pump current Ip3 and the second measurement pump current Ip6 flowing to the first measurement pump cell 60A and the second measurement pump cell 60B were measured (Experiment 2: refer to the second characteristic line L2 of FIG. 7, and the second section [2] of Table 1 of FIG. 8).

(9) Next, N2 and 3% of H₂O were made to flow as a base gas into the model gas measurement apparatus at 120 L/min, and the NO concentration was gradually reduced in a stepwise manner to NO=100, 80, 60, 40, 20, and 0 ppm, and with respect to each NO concentration of NO =80, 60, 40, 20, and 0 ppm, NH₃ was added to the gas, in a manner so as to maintain the second measurement pump current value Ip6 of the second measurement pump cell 60B at the time that NO=100 ppm at 2.137 μA. At this time, the flow rate of the base gas was adjusted so as to maintain the total gas flow rate at 120 L/min. In each respective gas atmosphere, the first measurement pump current Ip3 flowing to the first measurement pump cell 60A was measured (Experiment 3). The relationship between the respective concentrations of NO and NH₃, the first measurement pump current value Ip3 and the second measurement pump current value Ip6, and the difference (amount of change ΔIp) between the first measurement pump current value Ip3 and the second measurement pump current value Ip6 is shown by the first plot group P1 of FIG. 7, and the third section [3] of Table 1 of FIG. 8.

(10) Next, N2 and 3% of H₂O were made to flow as a base gas into the model gas measurement apparatus at 120 L/min, and the NO concentration was gradually reduced in a stepwise manner to NO=50, 40, 30, 20, 10, and 0 ppm, and with respect to each NO concentration of NO=40, 30, 20, 10, and 0 ppm, NH₃ was added to the gas, in a manner so as to maintain the second measurement pump current value Ip6 of the second measurement pump cell 60B at the time that NO=50 ppm at 1.070 μA. At this time, the flow rate of the base gas was adjusted so as to maintain the total gas flow rate at 120 L/min. In each respective gas atmosphere, the first measurement pump current Ip3 flowing to the first measurement pump cell 60A was measured (Experiment 4). The relationship between the respective concentrations of NO and NH₃, the first measurement pump current value Ip3 and the second measurement pump current value Ip6, and the difference (amount of change ΔIp) between the first measurement pump current value Ip3 and the second measurement pump current value Ip6 is shown by the second plot group P2 of FIG. 7, and the fourth section [4] of Table 1 of FIG. 8.

(11) Next, N2 and 3% of H₂O were made to flow as a base gas into the model gas measurement apparatus at 120 L/min, and the NO concentration was gradually reduced in a stepwise manner to NO=25, 20, 15, 10, 5, and 0 ppm, and with respect to each NO concentration of NO=20, 15, 10, 5, and 0 ppm, NH₃ was added to the gas, in a manner so as to maintain the second measurement pump current value Ip6 of the second measurement pump cell 60B at the time that NO=25 ppm at 0.537 μA. At this time, the flow rate of the base gas was adjusted so as to maintain the total gas flow rate at 120 L/min. In each respective gas atmosphere, the first measurement pump current Ip3 flowing to the first measurement pump cell 60A was measured (Experiment 5). The relationship between the respective concentrations of NO and NH₃, the first measurement pump current value Ip3 and the second measurement pump current value Ip6, and the difference (amount of change ΔIp) between the first measurement pump current value Ip3 and the second measurement pump current value Ip6 is shown by the third plot group P3 of FIG. 7, and the fifth section [5] of Table 1 of FIG. 8.

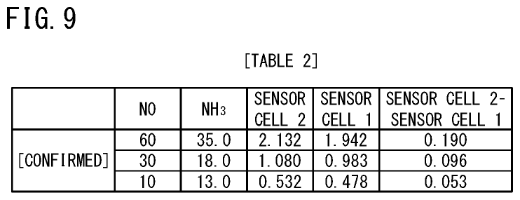

(12) Using the data obtained in Experiment 1 to Experiment 5, the map 110 shown in FIG. 7 corresponding to the first sensor cell 15A was created. In order to confirm the certainty of the obtained map 110, the first measurement pump current value Ip3 and the second measurement pump current value Ip6 in the mixed gases of NO and NH₃ having concentrations that differ from each other in Experiments 1 to 5, and the difference (amount of change ΔIp) between the first measurement pump current value Ip3 and the second measurement pump current value Ip6 were measured, whereupon the results shown in Table 2 of FIG. 9 were obtained. When the results of Table 2 (indicated by Δ) were plotted on the graph of FIG. 7, the results were in good agreement with the concentrations estimated from the map 110.

(13) Next, in a state in which the first switch SW1 is turned OFF, the second switch SW2 is turned ON, and the first preliminary adjustment pump cell 80A of the first sensor cell 15A is turned OFF, a state is brought about in which the second preliminary adjustment pump cell 80B of the second sensor cell 15B is turned ON. Thereafter, an experiment was carried out on the second sensor cell 15B with the same procedure as in the aforementioned items (1) to (5), and the voltage applied between the second preliminary pump electrode 82B and the exterior side pump electrode 46 was feedback-controlled, in a manner so that the electromotive force between the second preliminary pump electrode 82B and the reference electrode 52 of the second preliminary adjustment pump cell 80B became a predetermined voltage.

(14) Thereafter, the same experiments as in the aforementioned items (6) to (11) were carried out, and a map corresponding to the second sensor cell 15B was created. Since such a map has substantially the same content as the map 110 shown in FIG. 7, it was determined to use the map 110 as maps for both the first sensor cell 15A and the second sensor cell 15B.

Incidentally, for example, when only the first preliminary adjustment pump cell 80A is turned ON, electrode deterioration of only the first preliminary pump electrode 82A in the first preliminary adjustment pump cell 80A progresses. Thus, by switching the ON and OFF states of the first preliminary adjustment pump cell 80A and the second preliminary adjustment pump cell 80B, the durability of the first sensor cell 15A and the second sensor cell 15B as a whole is improved.

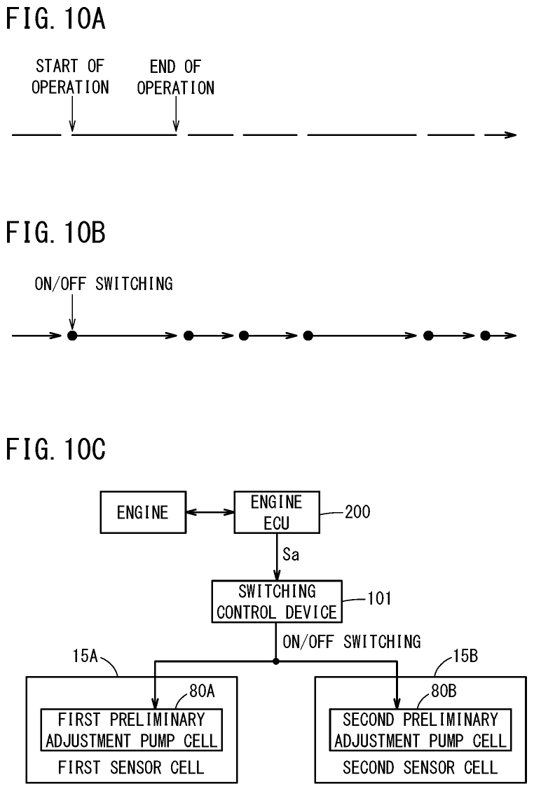

In this instance, a preferable switching timing (switching control) of the ON and OFF states of the first preliminary adjustment pump cell 80A and the second preliminary adjustment pump cell 80B will be described below with reference to FIGS. 10A to 17.

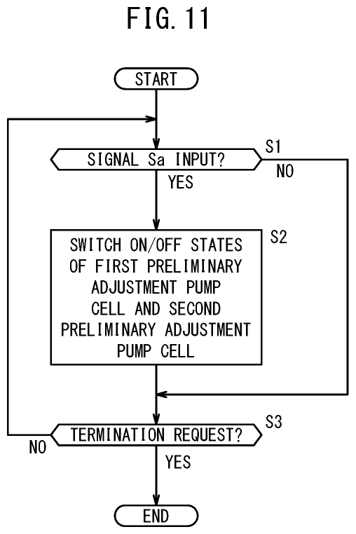

FIGS. 10A, 12A, 14A, and 16A are timing charts showing the start of operation and the end of operation of a vehicle or the like in which an engine is installed, and FIGS. 10B, 12B, 14B, and 16B are timing charts showing ON/OFF switching timings of the first preliminary adjustment pump cell 80A and the second preliminary adjustment pump cell 80B. Further, FIGS. 10C, 12C, 14C, and 16C are block diagrams of a switching control, and FIGS. 11, 13, 15, and 17 are flowcharts showing ON/OFF switching timings of the first preliminary adjustment pump cell 80A and the second preliminary adjustment pump cell 80B.

### [First Switching Timing]

As shown in FIGS. 10A and 10B, the first switching timing is a method of switching the ON and OFF states of the first preliminary adjustment pump cell 80A and the second preliminary adjustment pump cell 80B, at substantially the same time as starting operation of the drive source, for example, an engine (refer to FIG. 10C, etc.).

More specifically, as shown in FIG. 10C, the switching control device 101 realizes switching of the ON and OFF states of the first preliminary adjustment pump cell 80A and the second preliminary adjustment pump cell 80B, based on, for example, an engine operation start signal Sa from an engine ECU 200.

When an explanation is given based on the flowchart shown in FIG. 11, first, in step S1, the switching control device 101 determines, for example, whether or not the engine operation start signal Sa has been input from the engine ECU 200. In the case of having been input, then in step S2, the switching control device 101 switches the ON and OFF states of the first preliminary adjustment pump cell 80A and the second preliminary adjustment pump cell 80B.

For example, if the first preliminary adjustment pump cell 80A is turned ON and the second preliminary adjustment pump cell 80B is turned OFF, the first preliminary adjustment pump cell 80A is switched to OFF, and the second preliminary adjustment pump cell 80B is switched to ON. Of course, if the first preliminary adjustment pump cell 80A is turned OFF and the second preliminary adjustment pump cell 80B is turned ON, the first preliminary adjustment pump cell 80A is switched to ON, and the second preliminary adjustment pump cell 80B is switched to OFF. The same features apply in the descriptions given below.

Thereafter, in step S3, the switching control device 101 determines, for example, whether or not there is a termination request (interruption of power, maintenance, or the like) from the engine ECU 200. If there is no such termination request, then the processing from step S1 and thereafter is repeated, whereas if there is such a termination request, processing by the switching control device 101 is terminated.

### [Second Switching Timing]

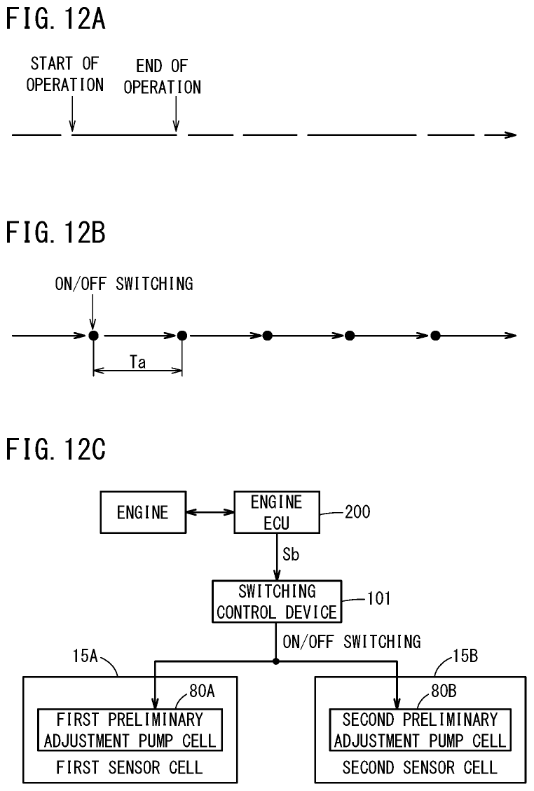

As shown in FIGS. 12A and 12B, the second switching timing is a method of switching the ON and OFF states of the first preliminary adjustment pump cell 80A and the second preliminary adjustment pump cell 80B, at each time that a fixed time period Ta which was set beforehand elapses, regardless of starting operation or ending operation of the engine.

More specifically, as shown in FIG. 12C, the switching control device 101 realizes switching of the ON and OFF states of the first preliminary adjustment pump cell 80A and the second preliminary adjustment pump cell 80B, based on, for example, a signal Sb indicating the elapse of the fixed time period Ta from the engine ECU 200.

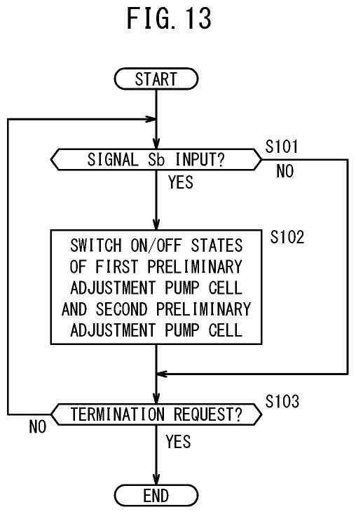

When an explanation is given based on the flowchart shown in FIG. 13, first, in step S101, the switching control device 101 determines, for example, whether or not the signal Sb indicating the elapse of the fixed time period Ta has been input from the engine ECU 200. In the case of having been input, then in step S102, the switching control device 101 switches the ON and OFF states of the first preliminary adjustment pump cell 80A and the second preliminary adjustment pump cell 80B.

Thereafter, in step S103, the switching control device 101 determines, for example, whether or not there is a termination request (interruption of power, maintenance, or the like) from the engine ECU 200. If there is no such termination request, then the processing from step S101 and thereafter is repeated, whereas if there is such a termination request, processing by the switching control device 101 is terminated.

### [Third Switching Timing]

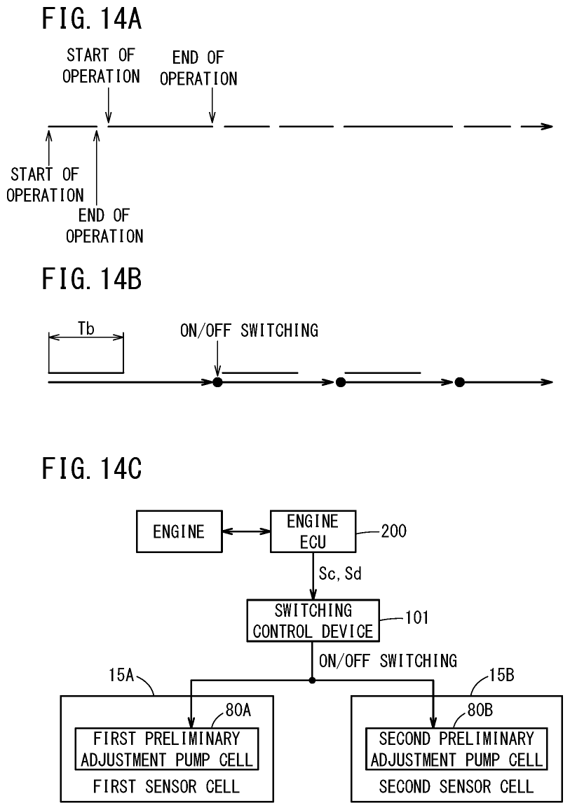

As shown in FIGS. 14A and 14B, the third switching timing is a method of switching the ON and OFF states of the first preliminary adjustment pump cell 80A and the second preliminary adjustment pump cell 80B, at a next time of starting operation, after a predetermined time period Tb has elapsed from having started operation.

More specifically, as shown in FIG. 14C, for example, based on a signal Sc from the engine ECU 200 indicating the elapse of the predetermined time period Tb from a point in time of having started operation, the switching control device 101 waits for input of a signal Sd indicating a start of next operation from the engine ECU 200. In addition, based on input of the signal Sd indicating the start of operation, switching of the ON and OFF states of the first preliminary adjustment pump cell 80A and the second preliminary adjustment pump cell 80B is realized.

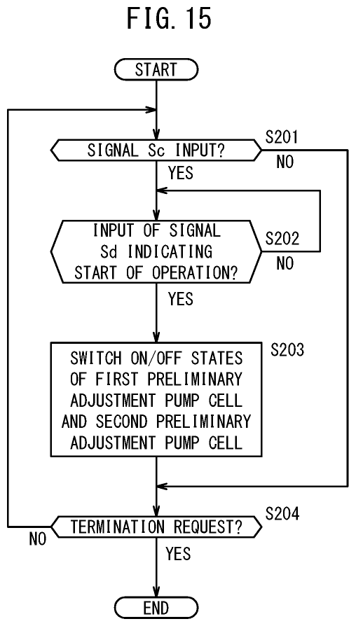

When an explanation is given based on the flowchart shown in FIG. 15, first, in step S201, the switching control device 101 determines, for example, whether or not the signal Sc indicating the elapse of the predetermined time period Tb from the point in time of having started operation has been input from the engine ECU 200. In the case of having been input, the process proceeds to step S202, and the switching control device 101 waits for input of the signal Sd indicating the start of next operation from the engine ECU 200. At a stage at which the signal Sd indicating the start of next operation is input, the process proceeds to step S203, and the switching control device 101 realizes switching of the ON and OFF states of the first preliminary adjustment pump cell 80A and the second preliminary adjustment pump cell 80B.

Thereafter, in step S204, the switching control device 101 determines, for example, whether or not there is a termination request (interruption of power, maintenance, or the like) from the engine ECU 200. If there is no such termination request, then the processing from step S201 and thereafter is repeated, whereas if there is such a termination request, processing by the switching control device 101 is terminated.

### [Fourth Switching Timing]

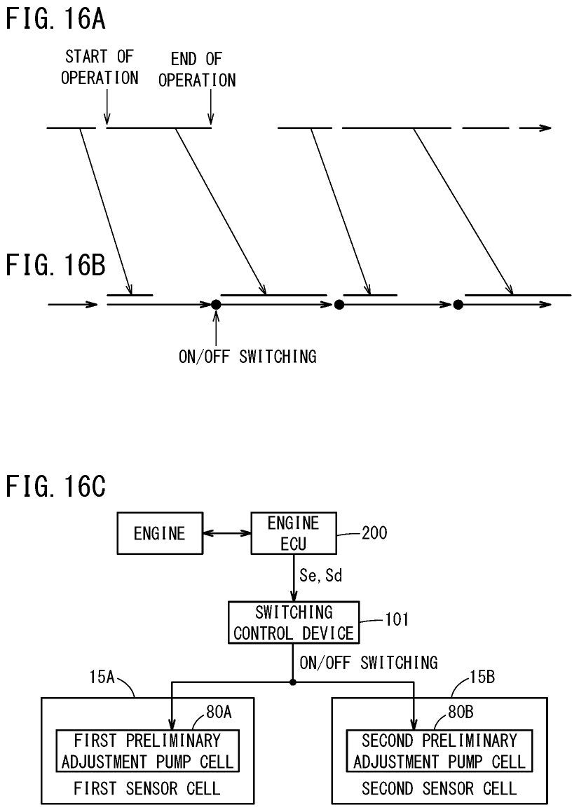

As shown in FIGS. 16A and 16B, although substantially the same as the aforementioned third switching timing, the fourth switching timing differs therefrom in that a previous operation time period is referred to, not after elapse of the predetermined time period Tb from the point in time of having started operation. More specifically, the fourth switching timing is a method of switching the ON and OFF states of the first preliminary adjustment pump cell 80A and the second preliminary adjustment pump cell 80B, at the time of starting next operation, after a same time period has elapsed as a previous operation time period from a point in time of having started the current operation.

More specifically, as shown in FIG. 16C, the engine ECU 200 retains the time period from the start of operation to the end of operation as the previous operation time period. In addition, a signal Se is output at a point in time when the previous operation time period has elapsed from the point in time of having started the current operation. Further, in a similar manner to the aforementioned third switching timing, the signal Sd indicating the start of next operation is output.

The switching control device 101, for example, based on input of the aforementioned signal Se from the engine ECU 200, waits for input of the signal Sd indicating the start of next operation from the engine ECU 200, and then, based on input of the signal Sd, realizes switching of the ON and OFF states of the first preliminary adjustment pump cell 80A and the second preliminary adjustment pump cell 80B.

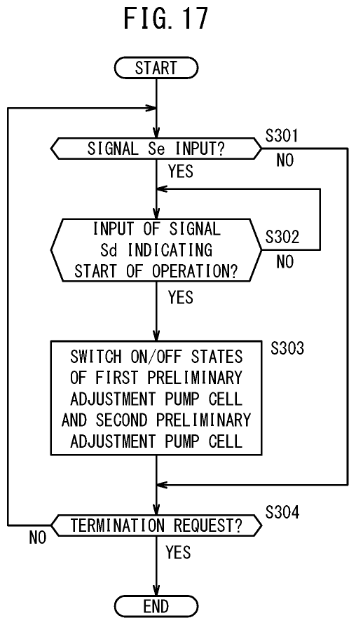

When an explanation is given based on the flowchart shown in FIG. 17, first, in step S301, the switching control device 101 determines, for example, whether or not the signal Se indicating the elapse of the previous operation time period from the point in time of having started the current operation has been input from the engine ECU 200. In the case of having been input, the process proceeds to step S302, and the switching control device 101 waits for input of the signal Sd indicating the start of next operation from the engine ECU 200. At a stage at which the signal Sd indicating the start of next operation is input, the process proceeds to step S303, and the switching control device 101 realizes switching of the ON and OFF states of the first preliminary adjustment pump cell 80A and the second preliminary adjustment pump cell 80B.

Thereafter, in step S304, the switching control device 101 determines, for example, whether or not there is a termination request (interruption of power, maintenance, or the like) from the engine ECU 200. If there is no such termination request, then the processing from step S301 and thereafter is repeated, whereas if there is such a termination request, processing by the switching control device 101 is terminated.

### Inventions Obtained from the Embodiment

The above-described embodiment can be summarized as follows.

[1] The gas sensor 10 according to the present embodiment is a gas sensor that measures concentrations of a first target component and a second target component, including:

the at least one sensor element (12, 12A, 12B);

the temperature control device (100) configured to control the temperature of the sensor element;

the at least one oxygen concentration control device (102A, 102B); and

the target component concentration acquisition device 104;

wherein the sensor element includes the structural body 14 made up from at least the oxygen ion conductive solid electrolyte, and the at least one sensor cell (15A, 15B) formed in the structural body 14;

the sensor cell is equipped, in a direction in which the gas is introduced, with the gas introduction port (16A, 16B), the first diffusion rate control member (34A, 34B), the first chamber (22A, 22B), the second diffusion rate control member (36A, 36B), the second chamber (18A, 18B), the third diffusion rate control member (38A, 38B), and the measurement chamber (20A, 20B);

the measurement chamber of the at least one sensor cell is equipped with the target component measurement pump cells (60A, 60B);

the oxygen concentration control device controls the oxygen concentrations of the first chamber and the second chamber of the at least one sensor cell; and

in the target component concentration acquisition device 104:

the concentration of the second target component is acquired on the basis of the difference between the current value flowing to one of the target component measurement pump cells, and the current value flowing to the other one of the target component measurement pump cells;

the total concentration of the first target component and the second target component is acquired from the current value flowing to the other one of the target component measurement pump cells; and

the concentration of the first target component is acquired by subtracting the concentration of the second target component from the total concentration.

In this manner, since the first oxygen concentration control device 102A controls the oxygen concentrations in the first chamber 22A and the second chamber 18A of the first sensor cell 15A, and the second oxygen concentration control device 102B controls the oxygen concentrations of the first chamber 22B and the second chamber 18B of the second sensor cell 15B, it is possible to measure the respective concentrations of a plurality of target components within the gas to be measured, while in addition, it is possible to accurately measure over a prolonged time period the concentration of a non-combustible component such as exhaust gas, and a plurality of components (for example, NO and NH₃) that coexist in the presence of oxygen.

Further, merely by changing the software of the control system of the gas sensor 10, the gas sensor 10 is capable of easily realizing the process of measuring the respective concentrations of NO and NH₃ which heretofore could not be realized, without separately adding various measurement devices or the like as hardware. As a result, it is possible to improve the accuracy of controlling a NOx purification system and detecting failures thereof. In particular, it is possible to distinguish between NO and NH₃ in the exhaust gas downstream of an SCR system, which contributes to precisely controlling the injected amount of urea, as well as detecting deterioration of the SCR system.

[2] In the present embodiment, the gas sensor may include one sensor element 12, and the sensor element 12 may include the first sensor cell 15A and the second sensor cell 15B. In accordance with such a configuration, by the one sensor element 12, it is possible to measure respective concentrations of a plurality of target components within the gas to be measured, and the size and scale of the measurement system can be reduced.

[3] In the present embodiment, the gas sensor may include two sensor elements 12A and 12B, one of the sensor elements 12A may include the first sensor cell 15A, and the other of the sensor elements 12B may include the second sensor cell 15B. In accordance with such a configuration, for example, it becomes possible for distal end portions of the first sensor element 12A and the second sensor element 12B (the first gas introduction port 16A and the second gas introduction port 16B) to be installed in facing relation to each other with respect to the measurement target location. Therefore, installation of the first sensor element 12A and the second sensor element 12B can be dealt with in a flexible manner.

[4] In the present embodiment, there is further provided:

the first preliminary adjustment pump cell 80A disposed on the side of the first chamber 22A of the first sensor cell 15A, and the first oxygen concentration adjustment pump cell 42A disposed on the side of the second chamber 18A of the first sensor cell 15A; and

the second preliminary adjustment pump cell 80B disposed on the side of the first chamber 22B of the second sensor cell 15B, and the second oxygen concentration adjustment pump cell 42B disposed on the side of the second chamber 18B of the second sensor cell 15B;

wherein the first oxygen concentration control device 102A is equipped with:

the first preliminary oxygen concentration control unit 108A configured to control the oxygen concentration of the first chamber 22A of the first sensor cell 15A by controlling the first preliminary adjustment pump cell 80A; and

the first oxygen concentration control unit 106A configured to control the oxygen concentration of the second chamber 18A of the first sensor cell 15A by controlling the first oxygen concentration adjustment pump cell 42A; and

wherein the second oxygen concentration control device 102B is equipped with:

the second preliminary oxygen concentration control unit 108B configured to control the oxygen concentration of the first chamber 22B of the second sensor cell 15B by controlling the second preliminary adjustment pump cell 80B; and

the second oxygen concentration control unit 106B configured to control the oxygen concentration of the second chamber 18B of the second sensor cell 15B by controlling the second oxygen concentration adjustment pump cell 42B.

In accordance with such a configuration, a gas sensor having a pump that is ON at all times, and a gas sensor having a pump that is OFF at all time are not used. More specifically, it is possible to avoid a problem in which electrode deterioration of the gas sensor having the pump which is ON at all times progresses more so than that of the other gas sensor whose pump is OFF at all time, and it is possible to realize lengthening of the useful lifetime of a gas sensor that is capable of measuring a plurality of components.

[5] In the present embodiment, there are further included the first switch SW1 configured to control driving of the first preliminary adjustment pump cell 80A to be turned ON or OFF, the second switch SW2 configured to control driving of the second preliminary adjustment pump cell 80B to be turned ON or OFF, and the switching control device 101 configured to control switching between the first switch SW1 and the second switch SW2.

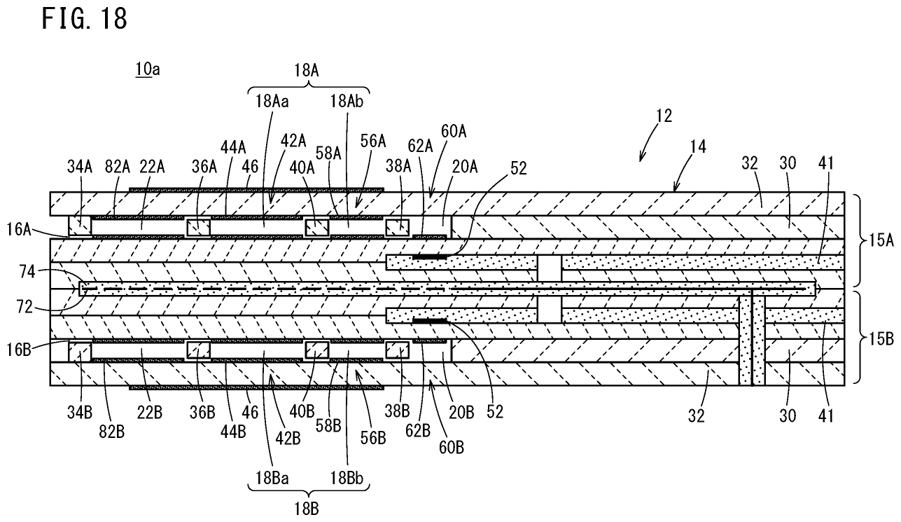

By controlling switching of the first switch SW1 and the second switch SW2 by the switching control device 101, it is possible to control driving, respectively, of the first preliminary adjustment pump cell 80A and the second preliminary adjustment pump cell 80B to be turned ON or OFF.

[6] In the present embodiment, the switching control device 101 may control switching between the first switch SW1 and the second switch SW2, substantially at the same time as starting operation of the drive source.

[7] In the present embodiment, the switching control device 101 may control switching between the first switch SW1 and the second switch SW2, at each time that the fixed time period Ta elapses, regardless of starting operation or ending operation of the drive source.

[8] In the present embodiment, the switching control device 101 may control switching between the first switch SW1 and the second switch SW2, at a time of starting next operation, after the predetermined time period Tb has elapsed from having started operation of the drive source. In this case, since variations in the ON time period are reduced, the present embodiment is effective in lengthening the useful lifetime of the gas sensor.

[9] In the present embodiment, the switching control device 101 may control switching between the first switch SW1 and the second switch SW2, at a time of starting next operation, after a same time period has elapsed as a previous operation time period from a point in time of having started operation of the drive source. In this case as well, since variations in the ON time period are reduced, the present embodiment is effective in lengthening the useful lifetime of the gas sensor.

[10] In the present embodiment, the second chamber 18A of the first sensor cell 15A may include the first main adjustment chamber 18Aa in communication with the first chamber 22A of the first sensor cell 15A, and the first auxiliary adjustment chamber 18Ab in communication with the first main adjustment chamber 18Aa, the second chamber 18B of the second sensor cell 15B may include the second main adjustment chamber 18Ba in communication with the first chamber 22B of the second sensor cell 15B, and the second auxiliary adjustment chamber 18Bb in communication with the second main adjustment chamber 18Ba, the first measurement chamber 20A of the first sensor cell 15A may be in communication with the first auxiliary adjustment chamber 18Ab, and the second measurement chamber 20B of the second sensor cell 15B may be in communication with the second auxiliary adjustment chamber 18Bb.

[11] In the present embodiment, the fourth diffusion rate control members 40A and 40B may be included, respectively, between the first main adjustment chamber 18Aa and the first auxiliary adjustment chamber 18Ab, and between the second main adjustment chamber 18Ba and the second auxiliary adjustment chamber 18Bb.

[12] In the present embodiment, the pump electrodes 82A and 82B may be included respectively in the first chamber 22A of the first sensor cell 15A, and the first chamber 22B of the second sensor cell 15B, the pump electrodes 44A and 44B may be included respectively in the second chamber 18A of the first sensor cell 15A, and the second chamber 18B of the second sensor cell 15B, the measurement electrodes 62A and 62B may be included respectively in the first measurement chamber 20A of the first sensor cell 15A, and the second measurement chamber 20B of the second sensor cell 15B, and each of the pump electrodes may be made of a material having a catalytic activity lower than that of the respective measurement electrodes.

[13] In the present embodiment, the first target component may be NO, and the second target component may be NH₃.

[14] In the present embodiment, the first preliminary oxygen concentration control unit 108A may control the oxygen concentration inside the first chamber 22A under a condition in which NH₃ is oxidized without causing decomposition of NO inside the first chamber 22A of the first sensor cell 15A, and the second preliminary oxygen concentration control unit 108B may control the oxygen concentration inside the second chamber 22B under a condition in which NH₃ is oxidized without causing decomposition of NO inside the second chamber 22B of the second sensor cell 15B.

[15] In the present embodiment, the target component concentration acquisition device 104 may utilize the map 110 in which there is specified the relationship between the NO concentration and the NH₃ concentration, respectively, by the current value Ip6, which is measured experimentally in advance, flowing to the second target component measurement pump cell 60B, and the difference ΔIp between the current value Ip3 flowing to the first target component measurement pump cell 60A and the current value Ip6 flowing to the second target component measurement pump cell 60B, and may determine the respective concentrations of NO and NH₃ by comparing with the map 110 the current value Ip6 flowing to the second target component measurement pump cell 60B during actual use, and the difference ΔIp between the current value Ip3 flowing to the first target component measurement pump cell 60A and the current value Ip6 flowing to the second target component measurement pump cell 60B.

[16] In the present embodiment, there may further be included the oxygen concentration detection device 70 configured to measure the oxygen concentration on the basis of the pump current value flowing to the second oxygen concentration adjustment pump cell 42B.

[17] In the present embodiment, a first exterior side pump electrode 46A disposed on the outer side of at least the second chamber 18A of the first sensor cell 15A, and a second exterior side pump electrode 46B disposed on the outer side of at least the second chamber 18B of the second sensor cell 15B may be provided in common. In accordance with this feature, the number of lead wires can be reduced, and mounting on various types of vehicles, for example, is facilitated.

[18] In the present embodiment, the first target component measurement pump cell 60A may include the first measurement electrode 62A disposed inside the first measurement chamber 20A of the first sensor cell 15A, and the first reference electrode 52 disposed in the reference gas introduction space 41 of the sensor element 12, the second target component measurement pump cell 60B may include the second measurement electrode 62B disposed inside the measurement chamber 20B of the second sensor cell 15B, and the second reference electrode 52 disposed in the reference gas introduction space 41 of the sensor element 12, and the first reference electrode 52 and the second reference electrode 52 (reference electrode 52 (see FIG. 1)) may be provided in common. In this case as well, the number of lead wires can be reduced, and mounting on various types of vehicles is facilitated.

[19] Moreover, as shown in the modification (gas sensor 10a) of FIG. 18, the first sensor cell 15A and the second sensor cell 15B may be disposed so as to substantially face each other in a thickness direction of the sensor element 12.

The gas sensor according to the present invention is not limited to the embodiments described above, and it is a matter of course that various configurations could be adopted therein without deviating from the essence and gist of the present invention.

In the example discussed above, in the first sensor cell 15A, the first measurement chamber 20A is disposed adjacent to the first auxiliary adjustment chamber 18Ab, and the first measurement electrode 62A is arranged inside the first measurement chamber 20A. However, apart therefrom, although not illustrated, the first measurement electrode 62A may be arranged inside the first auxiliary adjustment chamber 18Ab, and may be formed of a ceramic porous body such as alumina (Al₂O₃) serving as the third diffusion rate control member 38A, in a manner so as to cover the first measurement electrode 62A. In this case, the surrounding periphery of the first measurement electrode 62A functions as the first measurement chamber 20A.

The same features may also be applied to the second sensor cell 15B, and the second measurement electrode 62B may be arranged inside the second auxiliary adjustment chamber 18Bb, and may be formed of a ceramic porous body such as alumina (Al₂O₃) serving as the third diffusion rate control member 38B, in a manner so as to cover the second measurement electrode 62B. In this case, the surrounding periphery of the second measurement electrode 62B functions as the second measurement chamber 20B.

Further, in the above example, an example was illustrated in which NH₃ or NO₂ as the second target component is converted into NO inside the preliminary adjustment chambers 22A and 22B at a conversion ratio of 100%. However, the conversion ratio of NH₃ need not necessarily be 100%, and the conversion ratio can be set arbitrarily, within a range in which a correlation with good reproducibility with the NH₃ concentration within the gas to be measured is obtained.

Further, the respective operations of the first preliminary oxygen concentration control unit 108A and the second preliminary oxygen concentration control unit 108B may be performed in a direction of pumping oxygen out from, or in a direction of pumping oxygen into the interior of the first preliminary adjustment chamber 22A and the interior of the second preliminary adjustment chamber 22B, and it is sufficient insofar as the measurement pump currents Ip3 and Ip6, which are outputs of the measurement pump cell, change with good reproducibility due to the presence of NH₃ that serves as the second target component.

As shown in FIGS. 1 and 18, in the aforementioned gas sensors 10 and 10a, a structure is included in which a plurality of sensor cells (for example, the first sensor cell 15A and the second sensor cell 15B) are formed in a single structural body 14 constituting the sensor element 12.

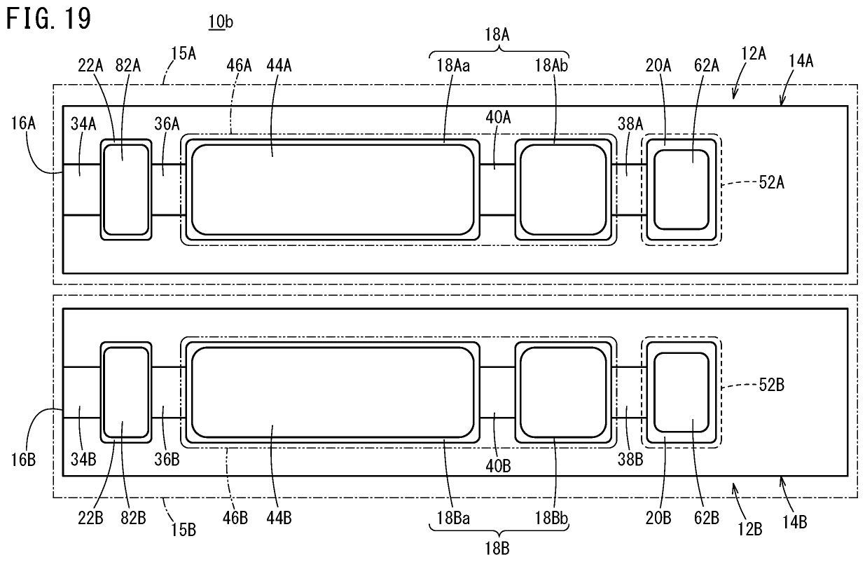

Apart therefrom, for example, as shown in FIG. 19, the gas sensor 10 may be provided which includes a plurality of sensor elements (for example, the first sensor element 12A and the second sensor element 12B). In the gas sensor 10 shown in FIG. 19, one first sensor cell 15A is formed in one first structural body 14A constituting the first sensor element 12A, and one second sensor cell 15B is formed in one second structural body 14B constituting the second sensor element 12B. Moreover, concerning the reference electrodes, a first reference electrode 52A is formed with respect to the first sensor element 12A, and a second reference electrode 52B is formed with respect to the second sensor element 12B.

Within the upper surface of the second solid electrolyte layer 32 of the first structural body 14A (see FIG. 2), the first exterior side pump electrode 46A is formed to extend from a region corresponding to the first main adjustment chamber 18Aa to a region corresponding to the first auxiliary adjustment chamber 18Ab. Similarly, within the upper surface of the second solid electrolyte layer 32 of the second structural body 14B (see FIG. 3), the second exterior side pump electrode 46B is formed to extend from a region corresponding to the second main adjustment chamber 18Ba to a region corresponding to the second auxiliary adjustment chamber 18Bb.

In practicing the present invention, various configurations for improving reliability may be added as components for an automotive vehicle, to such an extent that the concept of the present invention is not impaired.

What is claimed is:

1. A gas sensor configured to measure concentrations of a first target component and a second target component, comprising:
at least one sensor element;
a temperature control device configured to control a temperature of the sensor element;
at least one oxygen concentration control device; and
a target component concentration acquisition device;
wherein the sensor element includes a structural body made up from at least an oxygen ion conductive solid electrolyte, and at least one sensor cell formed in the structural body;
the sensor cell is equipped, in a direction in which a gas is introduced, with a gas introduction port, a first diffusion rate control member, a first chamber, a second diffusion rate control member, a second chamber, a third diffusion rate control member, and a measurement chamber;
the measurement chamber of the at least one sensor cell is equipped with target component measurement pump cells;
the oxygen concentration control device controls oxygen concentrations of the first chamber and the second chamber of the at least one sensor cell; and
in the target component concentration acquisition device:
a concentration of the second target component is acquired on a basis of a difference between a current value flowing to one of the target component measurement pump cells, and a current value flowing to another one of the target component measurement pump cells;
a total concentration of the first target component and the second target component is acquired from the current value flowing to the another one of the target component measurement pump cells; and
a concentration of the first target component is acquired by subtracting the concentration of the second target component from the total concentration.

2. The gas sensor according to claim 1, wherein the gas sensor includes one sensor element, and the sensor element includes a first sensor cell and a second sensor cell.

3. The gas sensor according to claim 1, wherein the gas sensor includes two sensor elements, one of the sensor elements includes a first sensor cell, and another of the sensor elements includes a second sensor cell.

4. The gas sensor according to claim 2, further comprising:
a first preliminary adjustment pump cell disposed on a side of the first chamber of the first sensor cell, and a first oxygen concentration adjustment pump cell disposed on a side of the second chamber of the first sensor cell; and
a second preliminary adjustment pump cell disposed on a side of the first chamber of the second sensor cell, and a second oxygen concentration adjustment pump cell disposed on a side of the second chamber of the second sensor cell;
wherein the first oxygen concentration control device is equipped with:
a first preliminary oxygen concentration control unit configured to control the oxygen concentration of the first chamber of the first sensor cell by controlling the first preliminary adjustment pump cell; and
a first oxygen concentration control unit configured to control the oxygen concentration of the second chamber of the first sensor cell by controlling the first oxygen concentration adjustment pump cell; and
wherein the second oxygen concentration control device is equipped with:
a second preliminary oxygen concentration control unit configured to control the oxygen concentration of the first chamber of the second sensor cell by controlling the second preliminary adjustment pump cell; and
a second oxygen concentration control unit configured to control the oxygen concentration of the second chamber of the second sensor cell by controlling the second oxygen concentration adjustment pump cell.

5. The gas sensor according to claim 4, further comprising:
a first switch configured to control driving of the first preliminary adjustment pump cell to be turned ON or OFF;
a second switch configured to control driving of the second preliminary adjustment pump cell to be turned ON or OFF; and
a switching control device configured to control switching between the first switch and the second switch.

6. The gas sensor according to claim 5, wherein the switching control device controls switching between the first switch and the second switch, substantially at same time as starting operation of the drive source.

7. The gas sensor according to claim 5, wherein the switching control device controls switching between the first switch and the second switch, at each time that a fixed time period elapses, regardless of starting operation or ending operation of the drive source.

8. The gas sensor according to claim 5, wherein the switching control device controls switching between the first switch and the second switch, at a time of starting next operation, after a predetermined time period has elapsed from having started operation of the drive source.

9. The gas sensor according to claim 5, wherein the switching control device controls switching between the first switch and the second switch, at a time of starting next operation, after a same time period has elapsed as a previous operation time period from a point in time of having started operation of the drive source.

10. The gas sensor according to claim 2, wherein:
the second chamber of the first sensor cell includes a first main adjustment chamber in communication with the first chamber of the first sensor cell, and a first auxiliary adjustment chamber in communication with the first main adjustment chamber;
the second chamber of the second sensor cell includes a second main adjustment chamber in communication with the first chamber of the second sensor cell, and a second auxiliary adjustment chamber in communication with the second main adjustment chamber;
the measurement chamber of the first sensor cell is in communication with the first auxiliary adjustment chamber; and
the measurement chamber of the second sensor cell is in communication with the second auxiliary adjustment chamber.

11. The gas sensor according to claim 10, wherein fourth diffusion rate control members are included, respectively, between the first main adjustment chamber and the first auxiliary adjustment chamber, and between the second main adjustment chamber and the second auxiliary adjustment chamber.

12. The gas sensor according to claim 2, wherein:
pump electrodes are included respectively in the first chamber of the first sensor cell, and the first chamber of the second sensor cell;
pump electrodes are included respectively in the second chamber of the first sensor cell, and the second chamber of the second sensor cell;
measurement electrodes are included respectively in the measurement chamber of the first sensor cell, and the measurement chamber of the second sensor cell; and
each of the pump electrodes is made of a material having a catalytic activity lower than that of the respective measurement electrodes.

13. The gas sensor according to claim 2, wherein the first target component is NO, and the second target component is NH₃.

14. The gas sensor according to claim 13, wherein the first preliminary oxygen concentration control unit controls the oxygen concentration inside the first chamber under a condition in which NH₃ is oxidized without causing decomposition of NO inside the first chamber of the first sensor cell; and
the second preliminary oxygen concentration control unit controls the oxygen concentration inside the second chamber under a condition in which NH₃ is oxidized without causing decomposition of NO inside the second chamber of the second sensor cell.

15. The gas sensor according to claim 13, wherein the target component concentration acquisition device:
utilizes a map in which there is specified a relationship between a NO concentration and a NH₃ concentration, respectively, by a current value, which is measured experimentally in advance, flowing to another one of the target component measurement pump cells, and a difference between a current value flowing to one of the target component measurement pump cells and the current value flowing to the another one of the target component measurement pump cells; and
determines the respective concentrations of NO and NH₃ by comparing with the map the current value flowing to the another one of the target component measurement pump cells during actual use, and the difference between the current value flowing to the one of the target component measurement pump cells and the current value flowing to the another one of the target component measurement pump cells.

16. The gas sensor according to claim 4, further including an oxygen concentration detection device configured to measure an oxygen concentration on a basis of a pump current value flowing to the second oxygen concentration adjustment pump cell.

17. The gas sensor according to claim 12, wherein a first exterior side pump electrode disposed on an outer side of at least the second chamber of the first sensor cell, and a second exterior side pump electrode disposed on an outer side of at least the second chamber of the second sensor cell are provided in common.

18. The gas sensor according to claim 2, wherein:
one of the target component measurement pump cells includes a first measurement electrode disposed inside the measurement chamber of the first sensor cell, and a first reference electrode disposed in a reference gas introduction space of the sensor element;
another one of the target component measurement pump cells includes a second measurement electrode disposed inside the measurement chamber of the second sensor cell, and a second reference electrode disposed in the reference gas introduction space of the sensor element; and
the first reference electrode and the second reference electrode are provided in common.

19. The gas sensor according to claim 2, wherein the first sensor cell and the second sensor cell are disposed so as to substantially face each other in a thickness direction of the sensor element.
